(19) United States
(12) Patent Application Publication
(10) Pub. No.: US 2007/0118151 A1
(43) Pub. Date: May 24, 2007

(54) Percutaneous cardiac valve repair with adjustable artificial chordae

(75) Inventor: Michael J. Davidson, Wellesley, MA (US)

Correspondence Address: EDWARDS & ANGELL, LLP, P.O. BOX 55874, BOSTON, MA, 02205, US

(73) Assignee: The Brigham and Women's Hospital, Inc.

(21) Appl. No.: 11/602,826

(22) Filed: Nov. 21, 2006

Related U.S. Application Data
(60) Provisional application No. 60/738,517, filed on Nov. 21, 2005.

(51) Int. Cl. A61B 17/138

(52) U.S. Cl. 606/144; 600/37; 606/151; 606/170

(57) ABSTRACT

The invention includes a novel method and system to achieve leaflet coaptation in a cardiac valve percutaneously by creation of neochordae to prolapsing valve segments. This technique is especially useful in cases of ruptured chordae, but may be utilized in any segment of prolapsing leaflet. The technique described herein has the additional advantage of being adjustable in the beating heart. This allows tailoring of leaflet coaptation height under various loading conditions using image-guidance, such as echocardiography. This offers an additional distinct advantage over conventional open-surgery placement of artificial chordae. In traditional open surgical valve repair, chord length must be estimated in the arrested heart and may or may not be correct once the patient is weaned from cardiopulmonary bypass. The technique described below also allows for placement of multiple artificial chordae, as dictated by the patient's pathophysiology.

### CROSS-REFERENCE TO RELATED APPLICATION

This application claims the benefit of priority from U.S. Provisional Patent Application Ser. No. 60/738,517 filed Nov. 21, 2005 the disclosure of which is incorporated by reference herein in its entirety.

### BACKGROUND OF THE INVENTION

1. Field of the Invention

The present invention relates to a system and method for treating the luminal system of a patient. Particularly, the present invention is directed to a system and method for treating the cardiac valves of a patient and also for adjusting the geometry of a patient's heart.

2. Description of Related Art

Mitral regurgitation (MR), or leakage, is the backflow of blood from the left ventricle into the left atrium due to an imperfect closure of the mitral valve. MR affects 5 in 10,000 persons in the United States. Myxomatous mitral degeneration has replaced rheumatic valve disease as the most common cause of surgically treated mitral regurgitation in developed nations. The primary pathophysiology in myxomatous mitral disease is prolapse of either the anterior and/or posterior leaflets of the mitral valve, leading to malcoaptation. The disease is also marked by dilation or deformation of the mitral annulus. The only effective treatment to reduce MR-related complications is by surgically repairing or replacing the mitral valve. The outcomes of mitral repair are significantly better than those for valve replacement, and this has become the procedure of choice in patients with clinically significant MR due to myxomatous disease. The mainstays of surgical repair approaches include both mitral valve annuloplasty and a procedure to reduce the height of the prolapsing leaflet(s) to improve leaflet coaptation. The options for the latter include leaflet resection, leaflet advancement, commissuroplasty, edge-to-edge plication, chordal transfer, and creation of neochordae. Each of these approaches has advantages and disadvantages and are often used in combination. The results of open surgical mitral valve repair for myxomatous MR are excellent, with greater than 90% freedom from reoperation at 10 years.

In general, a relatively significant gap may exist between the anterior leaflet and posterior leaflet of the mitral valve for a variety of different reasons. For example, a gap may exist due to congenital malformations, because of ischemic disease, or because a heart has been damaged by a previous heart attack. A gap may also be created when congestive heart failure, e.g., cardiomyopathy, or some other type of distress causes a heart to be enlarged. When a heart is enlarged, the walls of the heart, e.g., wall of a left ventricle, may stretch or dilate, causing the posterior leaflet of the mitral valve to stretch or be displaced. Accordingly, a gap can be created between the leaflets of the mitral valve when the walls of the left ventricle stretch. Hence, due to the existence of the gap, the mitral valve is unable to close properly, and may begin to leak. Leakage through the mitral valve generally causes a heart to operate less efficiently, as the heart must work harder to maintain a proper amount of blood flow therethrough.

Treatments used to correct for mitral valve leakage are typically highly invasive, open-heart surgical procedures. Ventricular assist devices such as artificial hearts may be implanted in a patient whose own heart is failing. The implantation of a ventricular assist device is often expensive, and a patient with a ventricular assist device must be placed on extended anti-coagulant therapy. As will be appreciated by those skilled in the art, anti-coagulant therapy reduces the risk of blood clots being formed, as for example, within the ventricular assist device. While reducing the risks of blood clots associated with the ventricular assist device is desirable, anti-coagulant therapies may increase the risk of uncontrollable bleeding in a patient, e.g., as a result of a fall, which is not desirable.

Open-heart surgical procedures which are intended to correct for mitral valve leakage, specifically, involve the implantation of replacement valves. Valves from animals, e.g., pigs, may be used to replace a mitral valve in a human. While the use of a pig valve may relatively successfully replace a mitral valve, such valves generally wear out, thereby requiring additional open surgery at a later date. Mechanical valves, which are less likely to wear out, may also be used to replace a leaking mitral valve. However, when a mechanical valve is implanted, there is an increased risk of thromboembolism, and a patient is generally required to undergo extended anti-coagulant therapies.

One open-heart surgical procedure that is particularly successful in correcting for mitral valve leakage is an annuloplasty procedure. During an annuloplasty procedure, an annuloplasty ring may be implanted on the mitral valve to cause the size of a stretched mitral valve to be reduced to a relatively normal size. An annuloplasty ring is shaped approximately like the contour of a normal mitral valve. That is, an annuloplasty ring is shaped substantially like the letter “D.” Typically, annuloplasty rings may be formed from a rod or tube of biocompatible material, e.g., plastic, that as a DACRON mesh covering.

In order for an annuloplasty ring to be implanted, a surgeon surgically attaches the annuloplasty ring to the mitral valve on the atrial side of the mitral valve. Conventional methods for installing such a ring require open-heart surgery which involve opening a patient's sternum and placing the patient on a heart bypass machine. The annuloplasty ring is sewn to the posterior mitral annulus and the fibrous trigones at the top portion of the mitral valve. In sewing the annuloplasty ring onto the mitral valve, a surgeon generally alternately acquires a relatively large amount of tissue from mitral tissue, e.g., a one-eighth inch bite of tissue, using a needle and thread, followed by a smaller bite from the annuloplasty ring. Once a thread has loosely coupled the annuloplasty ring to the mitral valve tissue, the annuloplasty ring is slid onto the mitral valve such that tissue that was previously stretched out, e.g., due to an enlarged heart, is effectively pulled in using tension applied by the annuloplasty ring and the thread which binds the annuloplasty ring to the mitral valve tissue. As a result, the gap between the anterior leaflet and the posterior leaflet may be substantially closed off. After the mitral valve is shaped by the annuloplasty ring, the anterior and posterior leaflets of the mitral valve will reform to create a new coaptation surface and will enable the mitral valve to appear and to function as a normal mitral valve.

Once implanted, tissue generally grows over the annuloplasty ring, and a line of contact between the annuloplasty ring and the mitral valve will essentially enable the mitral valve to appear and function as a normal mitral valve. Although a patient who receives the annuloplasty ring may be subjected to anti-coagulant therapies, the therapies are not extensive, as a patient is only subjected to the therapies for a matter of weeks, e.g., until tissue grows over the annuloplasty ring.

A second surgical procedure which is generally effective in reducing mitral valve leakage involves placing an edge-to-edge suture in the mitral valve. Such a surgical procedure, e.g., an Alfieri stitch procedure or a bow-tie repair procedure, will be described. An edge-to-edge stitch is used to stitch together an area at approximately the center of a gap defined between the anterior and posterior leaflets of the mitral valve. Once the stitch is in place, the stitch is pulled in to form a suture which holds anterior leaflet against the posterior leaflet, as shown. By reducing the size of the gap between the anterior leaflet and the posterior leaflet, the amount of leakage through the mitral valve may be substantially reduced.

Although the placement of an edge-to-edge stitch is generally successful in reducing the amount of mitral valve leakage through the gap between the leaflets of the mitral valve, this technique is conventionally made through open-heart surgery. In addition, the use of the edge-to-edge stitch is generally not suitable for a patient with an enlarged, dilated heart, as blood pressure causes the heart to dilate outward, and may put a relatively large amount of stress on the edge-to-edge stitch.

While invasive surgical procedures have proven to be effective in the treatment of mitral valve leakage, invasive surgical procedures often have significant drawbacks. Any time a patient undergoes open-heart surgery, there is a risk of infection. Opening the sternum and using a cardiopulmonary bypass machine has also been shown to result in a significant incidence of both short and long term neurological deficits.

It is also possible to address mitral valve regurgitation by anchoring artificial chordae between the posterior leaflet of the mitral valve and papillary muscles in the left ventricle. In accordance with this procedure, a length of non-resorbable suture (e.g., expanded PTFE) is sutured between the two locations in an effort to make the anterior and posterior mitral valve leaflets realign, reducing regurgitation. However, this procedure suffers from certain disadvantages. First, as with other open heart surgical techniques, such procedures require that the patient's heart be stopped in order to place the sutures. Since the heart is stopped when the suture is installed, the surgeon has to estimate the length of the suture that needs to be used. After the patient is removed from bypass and the patient's heart is restarted, it is entirely possible that the length of the suture will be incorrect, resulting in no improvement to the patient's condition. This can require stopping the patient's heart again to repeat the procedure, which carries the added risk that the patient's heart might not restart.

Thus, there still remains a continued need in the art for improved surgical techniques for treating mitral valve regurgitation. The present invention provides a solution for these problems, as described herein.

### SUMMARY OF THE INVENTION

The purpose and advantages of the present invention will be set forth in and apparent from the description that follows, as well as will be learned by practice of the invention. Additional advantages of the invention will be realized and attained by the methods and systems particularly pointed out in the written description hereof, as well as from the appended drawings.

To achieve these and other advantages and in accordance with the purpose of the invention, as embodied herein and broadly described, in accordance with one aspect, the invention includes a method of repairing a cardiac valve. The method includes introducing a catheter through a patient's vasculature into the patient's heart and advancing a distal end of the catheter proximate a leaflet of a cardiac valve of the patient. The catheter is then used to direct a first portion of a filament through the leaflet to capture the leaflet. Tension is then applied to the filament to adjust the function of the cardiac valve.

In accordance with further aspects of the invention, the method may further include adjusting a length of the filament to vary the function of the cardiac valve. The length of the filament may be adjusted while the patient's heart is beating. If desired, the patient's heart may be viewed using an imaging technique while the length of the filament is adjusted to determine a desired length for the filament.

In further accordance with the invention, a second portion of the filament may be directed toward a second location within the patient's vasculature. The second portion of the filament may be directed to the second location, for example, by implanting an anchor in cardiac tissue in the second location, and directing the second portion of the filament through a portion of the anchor. If desired, the length of the filament may be adjusted by applying tension to the filament through the anchor. Moreover, tension may be applied to the filament by disposing a distal end of the catheter against the anchor and pulling on the filament using the anchor as a fulcrum.

In accordance with a further aspect of the invention, the method may further include applying a lock to the filament to prevent the second portion of the filament from disengaging from the anchor. The lock may be applied to a portion of the filament that is on an opposite side of the anchor from the first portion of the filament. The filament is preferably severed on a side of the lock opposite the anchor. If desired, the lock may be applied to the filament using the same catheter used to deliver the filament to the valve leaflet. By way of further example, the filament may be severed using the same catheter used to deliver the filament to the leaflet.

In accordance with still another aspect of the invention, the filament includes suture material. The suture material may include a monofilament and/or a polyfilament braided material. By way of further example, the suture material may include material selected from the group consisting of polypropylene, polyester, nylon, and silk, among others. The suture material may also include radiopaque material. The suture material may additionally or alternatively have echodense properties to facilitate visualization of the filament using fluoroscopic or echocardiographic imaging techniques. The imaging technique may be selected, for example, from the group including echocardiography and fluoroscopy.

If desired, the suture material may include expanded PolyTetraFluoroEthylene (“ePTFE”). Accordingly, the ePTFE suture material may include nodes and fibrils adapted to facilitate tissue ingrowth therein.

In accordance with still a further aspect of the invention, the anchor may include at least one barb, the barb being adapted and configured to resist backout of the anchor from tissue in which the anchor is implanted. The barb may be deployed from an undeployed to a deployed position after implanting the anchor in tissue of the patient.

In accordance with yet further aspects of the invention, the catheter may be introduced into the patient's vasculature through a guide catheter. Moreover, the catheter may additionally or alternatively be introduced into the patient's vasculature over a guidewire.

In accordance with still further aspects of the invention, the methods and systems embodied herein may be used to perform a procedure on a patient's mitral valve. If desired, a first end of a filament may be placed on a patient's mitral valve leaflet, and a second portion of the filament may be affixed to a papillary muscle portion of the patient. In accordance with a further example, the patient's tricuspid valve may be treated using the methods and systems embodied herein. The method may further include using a catheter to direct a first portion of a second filament through a valve leaflet to capture the leaflet, and applying tension to the second filament to further adjust the function of the cardiac valve being operated on.

In accordance with a further aspect of a method of the invention, a second portion of the filament may be affixed to a first portion of a second filament. A second portion of the second filament may accordingly be attached to an anchoring location within the patient's heart. The second filament may be attached to the anchoring location by affixing it to an anchor embedded in cardiac tissue. In accordance with one embodiment, the anchor may be embedded in a papillary muscle. If desired, the anchor may be implanted by the same catheter used to deliver the filament. If desired, the filaments may be affixed to each other using the same catheter used to deliver the filament to the valve leaflet.

The invention also provides a method of adjusting the geometry of a patient's heart. The method includes introducing a catheter through a patient's vasculature into the patient's heart and advancing a distal end of the catheter proximate a first portion of the interior of the patient's heart. The catheter is then used to attach a first portion of a filament to the first portion, and to attach a second portion of the filament to a second portion of the interior of the patient's heart. Tension is then applied to the filament to adjust the geometry of the patient's heart.

In accordance with further aspects of the invention, the final length of the filament may be set. For example, the final length of the filament may be set by applying a retainer to the filament. If desired, the geometry of the patient's heart may be adjusted to decrease mitral valve regurgitation. The mitral valve regurgitation may be decreased by reducing the septal-lateral dimension of the patient's ventricle. By way of further example, the geometry of the patient's heart may be adjusted to improve operation of the patient's tricuspid valve.

The invention also provides a catheter adapted and configured to deliver a filament through a patient's vasculature into the patient's heart proximate a leaflet of a cardiac valve of the patient. The catheter includes an elongate body having a proximal end and a distal end and a filament in operable association with the elongate body. The catheter further includes a deployable penetrator in operable association with the elongate body, the deployable penetrator being adapted and configured to be deployed through a portion of a patient's valve leaflet to facilitate capture of the leaflet by the filament.

In further accordance with the invention, the catheter may include a leaflet grasping portion adapted and configured to hold the patient's valve leaflet in place to facilitate deploying the penetrator through the leaflet. The leaflet grasping portion is preferably adapted and configured to hold the patient's valve leaflet in place at least in part by applying suction to the valve leaflet. If desired, the leaflet grasping portion may be pivotally mounted proximate a distal region of the catheter. If further desired, the leaflet grasping portion of the catheter may be provided with two pivotally mounted arms mounted on a distal region of the catheter that are adapted and configured to grasp a valve leaflet along opposing faces of the leaflet. If further desired, the catheter may further include a second filament adapted and configured to receive the penetrator. The second filament may include a cuff adapted and configured to receive the penetrator. If desired, the second filament may further include a loop formed therein for receiving the first filament therethrough.

In accordance with a further aspect of the invention, the catheter may be adapted and configured to permit the second filament to be pulled through the leaflet to permit the loop to form a knot about the leaflet to capture the leaflet. The catheter may be further adapted and configured to permit a free end of the second filament to be exteriorized from the patient. Preferably, the second filament is exteriorized through a lumen of the catheter.

In accordance with yet a further aspect of the invention, the catheter may further include an anchor deployment portion for deploying an anchor into cardiac tissue of the patient. Moreover, the catheter may further include a retainer applicator for applying a retainer to the filament. The retainer applicator is preferably adapted to apply a retainer to the filament proximate an anchor embedded in the patient's vasculature. By way of further example, the retainer applicator may be adapted to apply a retainer to a plurality of filaments to secure the plurality of filaments to each other. In accordance with a further embodiment of the invention, the catheter may further include a blade disposed thereon adapted and configured to severe the filament.

The invention also provides a catheter for applying an anchor into cardiac tissue of a patient. The catheter includes an elongate outer body having a proximal end, a distal end and defining a lumen therethrough. The catheter further includes a torquable elongate inner body movably disposed within the lumen of the outer body, the inner body having a fitting for receiving an anchor therein.

In accordance with a further aspect of the invention, the inner body may be formed at least in part from a hypotube. If desired, the inner body and/or outer body may have a varying flexibility along their lengths. The varying flexibility may be provided at least in part by a plurality of cuts formed in the inner body, and/or by at least one stiffening wire formed into the outer body. If desired, a guide lumen may be formed on the outer body having a filament inlet port proximate the distal end of the outer body and a filament exit port proximal of the filament inlet port for receiving a filament therethrough. Preferably, the filament exit port is substantially close to the distal end of the outer body.

In accordance with still further aspects of the invention, the catheter may further include an anchor disposed in the fitting, and a filament disposed about the inner body, the filament being operatively associated with the anchor. A torquable handle may be attached to the proximal end of the inner body for applying a torque to the inner body, and the anchor. If desired, the catheter may further include a steering mechanism adapted and configured to steer a distal end of the catheter. Moreover, an anchor guide may be disposed on an inside surface of the outer body, the anchor guide being adapted and configured to guide the anchor during installation of the anchor. If desired, the anchor may be a helically shaped anchor that is disposed in the fitting, wherein the anchor guide facilitates rotation of the anchor while it is being installed. Accordingly, the anchor guide preferably urges the anchor distally as it is rotated with respect to the outer body by the inner body. The anchor may include at least one barb disposed thereon to resist backout of the anchor after it has been implanted. The anchor may be formed from shape memory material, among other materials.

The invention also provides a catheter for fastening together a plurality of filaments. The catheter includes an elongate body having a proximal end and a distal end, and a guide passage for receiving a plurality of filaments to be fastened together. The catheter further includes a fastener applicator disposed proximate the distal end of the elongate body for applying a fastener to the plurality of filaments received by the guide passage. The catheter may further include an actuator disposed proximate the proximal end of the elongate body operably coupled to the fastener applicator to facilitate fastening the plurality of filaments.

In further accordance with the invention, the guide passage of the catheter may include a distal opening proximate the distal end of the elongate body for receiving the filaments, and a proximal exit opening spaced proximally from the distal end of the elongate body that permits passage of the filaments therethrough. The proximal exit opening may be substantially closer to the distal end of the elongate body than the proximal end of the elongate body.

The invention further provides a catheter for positioning a lock on a filament. The catheter includes an inner member having a proximal end, a distal end and defining a lumen at least partially therethrough, the lumen being adapted and configured to receive a filament therethrough. The catheter further includes a filament lock biased to change from a first, relatively open state to a second, relatively closed state, the filament lock being disposed on a substantially rigid portion of the inner member, the inner member being sufficiently rigid to prevent the filament lock from changing from the first state to the second state. The catheter further includes an outer member having a proximal end, a distal end and defining a lumen at least partially therethrough. The lumen of the outer member is adapted and configured to movably receive the inner member. The lumen of the outer member is preferably sufficiently small to prevent the filament lock from entering the lumen when positioned on the inner member.

In further accordance with the invention, the outer member can be used to urge the lock distally off of the inner member and onto the filament. If desired, the filament lock may include a helical body wound about the inner member biased to contract in radius from the first state to the second state. The filament lock may include a pair of jaws hingedly connected that are biased to close on the filament. By way of further example, the filament lock may include a plurality of legs that are biased to close on the filament. If desired, the filament lock may include a substantially tubular body made from shape memory material that is adapted and configured to contract about the filament when exposed to the temperature of the body of the patient. If desired, the catheter may further include a pair of jaws adapted and configured to crimp the filament lock on the filament, and an actuator for actuating the jaws. The catheter may further include a filament exit port disposed in each of the inner member and outer member to permit passage of the filament therethrough. The filament exit ports are preferably proximate the distal end of the inner and outer members.

The invention further provides an anchor adapted and configured to be anchored in cardiac tissue of a patient. The anchor includes an anchoring portion having a proximal end and a distal end, the anchoring portion being adapted and configured to be anchored into cardiac tissue of a patient. The anchor further includes a filament lock disposed at the proximal end of the anchoring portion.

In accordance with a further aspect of the invention, the filament lock is preferably biased to change from a first, relatively open state to a second, relatively closed state when disposed about a filament. The filament lock preferably defines a lumen therethrough for receiving an locking onto a filament. However, filament locks as disclosed herein may include a substantially planar member that is caused to deform to surround and clamp a filament. The anchor may be formed, for example, at least in part from a shape memory material. The shape memory material may include nitinol.

In still further accordance with the invention, the anchor may further include a coupling member affixed to the anchoring portion, the coupling member defining a lumen therethrough for receiving a filament.

The invention also provides a catheter for severing a filament inside of a patient's vasculature. The catheter includes an inner member having a proximal end, a distal end, and defining a lumen therethrough, the lumen being adapted and configured to receive a filament therethrough. The catheter also includes an outer member having a proximal end, a distal end and defining a lumen therethrough, the lumen of the outer member being adapted and configured to receive the inner member. The catheter also includes a pair of substantially arcuate cutting jaws pivotally mounted inside the lumen of the outer member in a wall of the outer member.

In further accordance with the invention, the jaws are preferably biased to close about and sever the filament. The jaws may be held apart by the inner member when the inner member is positioned between the jaws, wherein the filament is severed when the inner member is moved out of alignment with the cutting jaws.

In further accordance with the invention, a method of treating a cardiac valve is provided. The method includes introducing a catheter through a patient's vasculature into the patient's heart, and advancing a distal end of the catheter proximate a leaflet of a cardiac valve of the patient. The method further includes using the catheter to direct a first portion of a filament through the leaflet to capture the leaflet, and attaching a second end of the filament to cardiac tissue proximate an annulus of the cardiac valve. Tension is then applied to the filament to cause the leaflet of the cardiac valve to fold over onto itself until coaptation of the leaflet is established with an adjoining valve leaflet.

In further accordance with the invention, the leaflet may be attached to the annulus of the cardiac valve using a plurality of connected filaments joined by a retainer.

The invention also provides a system for treating a cardiac valve of a patient. The system includes a first catheter for directing a first portion of a filament through a leaflet of a patient's cardiac valve to capture the leaflet, and a second catheter for implanting an anchor into cardiac tissue of the patient displaced from the leaflet. These and other elements can be packaged and sold as a kit with instructions for use, if desired.

In further accordance with the invention, the system may further include a third catheter for applying a filament lock to the filament after tension has been applied to the filament to change the operation of the cardiac valve. The second catheter may be adapted and configured to receive the filament from the first catheter and direct the filament through the anchor. The third catheter may further include a blade for severing the filament after the filament lock has been applied. If desired, the filament may include suture material. The suture material may include material selected from the group consisting of polypropylene, polyester, nylon, silk and expanded PolyTetraFluoroEthylene, among others.

The invention also provides a method of implanting a filament in a lumenal system of a patient. The method includes introducing a catheter through a lumenal system of a patient to a location in the patient to be treated. The method further includes advancing a distal region of the catheter proximate a first location and attaching a first portion of a filament to the first location using the catheter. The method further includes advancing the distal region of the catheter proximate a second location and attaching a second portion of the filament to the second location using the catheter.

In accordance with a further aspect of the invention, the location to be treated is preferably inside of the patient's heart. The filament may have a predetermined length established outside of the body of the patient. The method may further include determining a length of the filament outside of the body of the patient prior to introducing the catheter into the lumenal system of the patient. The length of the filament may be determined by using an imaging technique. The imaging technique may be selected from the group consisting of echocardiography and fluoroscopy, among others.

In accordance with still a further aspect of the invention, the first region may be a valve leaflet. The second region may be a papillary muscle. The valve leaflet may be located on the patient's tricuspid valve.

The invention also provides a system for implanting a filament in a lumenal system of a patient. The system includes a catheter having an elongate body, the elongate body having a proximal end and a distal end, and an elongate filament. The filament has a predetermined length. The filament further has a first means for attachment to tissue at a first portion thereof and a second means for attachment to tissue at a second portion thereof.

In further accordance with the invention, the first means for attachment may be adapted and configured to connect the first portion of the filament to a valve leaflet of a patient. The second means for attachment may be adapted and configured to connect the second portion of the filament to cardiac tissue of a patient. The cardiac tissue may be a papillary muscle head of the patient. If desired, at least one of the first means for attachment and second means for attachment may include at least one barb for anchoring into tissue.

The techniques described herein are especially useful in cases of ruptured chordae, but may be utilized in any segment of prolapsing leaflet. Many of the techniques described herein have the additional advantage of being adjustable in the beating heart. This allows tailoring of leaflet coaptation height under various loading conditions using image-guidance, such as echocardiography. This offers an additional distinct advantage over conventional open-surgery placement of artificial chordae. In traditional open surgical valve repair, chord length must be estimated in the arrested heart and may or may not be correct once the patient is weaned from cardiopulmonary bypass. The technique described below also allows for placement of multiple artificial chordae, as dictated by the patient's pathophysiology. The methods and systems herein are also suitable for treating other cardiovascular disorders, such as tricuspid valve regurgitation, for example, and as described herein.

It is to be understood that both the foregoing general description and the following detailed description are exemplary and are intended to provide further explanation of the invention claimed.

The accompanying drawings, which are incorporated in and constitute part of this specification, are included to illustrate and provide a further understanding of the method and system of the invention. Together with the description, the drawings serve to explain the principles of the invention.

### BRIEF DESCRIPTION OF THE DRAWINGS

FIG. 1 is a schematic view of a first representative embodiment of a medical device made in accordance with the teachings of the present invention and an associated method.

FIGS. 2-4 are views of further aspects of the device and method depicted in FIG. 1.

FIGS. 5-10 are schematic views of another medical device made in accordance with the teachings of the present invention illustrating a representative method of valve leaflet capture.

FIGS. 11-15 are schematic views of a representative embodiment of an anchor delivery catheter made in accordance with the invention and illustrations of an associated exemplary method of delivering an anchor to a location within a patient's vasculature.

FIG. 16 is a further schematic depiction of a method carried out in accordance with the invention.

FIG. 17 is an illustration of a representative embodiment of a catheter for attaching a plurality of sutures to one another in accordance with the present invention.

FIGS. 18-19 are further schematic depictions of a method carried out in accordance with the invention.

FIGS. 20-22 are schematic depictions of portions of another method carried out in accordance with the invention.

FIGS. 23-24 are schematic depictions of representative anchors made in accordance with the present invention.

FIGS. 25-27 are schematic depictions of another system and method for deploying an anchor and filament in accordance with the present invention

FIGS. 28-29 illustrate further aspects of anchors made in accordance with the present invention and the manner in which the anchors can be retained in an associated delivery device made in accordance with the present invention.

FIGS. 30-31 illustrate further embodiments of anchors made in accordance with the present invention.

FIGS. 32(a)-32(b) illustrate a representative embodiment of a suture or filament lock and associated delivery system made in accordance with the invention.

FIGS. 33-35 illustrate further representative embodiments of a suture or filament lock and portions of an associated delivery system made in accordance with the invention.

FIGS. 36(a)-36(e) illustrate a representative embodiments of suture severing devices made in accordance with the present invention in various operating conditions.

FIGS. 37-42 depict an additional representative method and system carried out in accordance with the teachings of the present invention.

FIGS. 43-49 depict yet an additional representative method and system carried out in accordance with the teachings of the present invention.

FIGS. 50(a)-50(c) depict views of various neochordae structures made in accordance with the present invention.

FIGS. 51-56 depict still additional representative methods and systems carried out in accordance with the teachings of the present invention for capturing a valve leaflet.

FIGS. 57-60 depict additional representative methods and systems carried out in accordance with the teachings of the present invention for anchoring an anchor and suture in cardiac tissue of a patient.

FIGS. 61-63 depict still additional embodiments of methods and systems carried out in accordance with the teachings of the present invention for capturing various cardiac tissue with an anchor.

FIGS. 64-68 depict further representative methods and systems for altering the geometry of a patient's heart in accordance with the present invention.

FIGS. 69-73 depict illustrations of an aspect of a representative method carried out in accordance with the teachings of the present invention.

FIGS. 74(a)-74(f) depict illustrations of still a further representative method carried out in accordance with the teachings of the present invention.

FIGS. 75(a)-75(c) are representative schematic views of another representative of a device and associated method in accordance with the present invention.

### DETAILED DESCRIPTION OF PREFERRED EMBODIMENTS

Reference will now be made in detail to the present preferred embodiments of the invention, examples of which are illustrated in the accompanying drawings. The methods and corresponding steps of the invention will be described in conjunction with the detailed description of the system.

The systems and methods provided in accordance with the teachings of the present invention allow adjustment of the geometry of various portions of a patient's heart. For example, systems and methods in accordance with the invention permit creation of an artificial mitral valve chord from a ventricular papillary muscle to the valve leaflet by a percutaneous approach and without the use of cardiopulmonary bypass or any need to stop the heart of the patient. The length of this artificial chord can be adjustable until the device used to implant the chord is uncoupled. By way of further example, neochordae of predetermined lengths can also be implanted percutaneously in accordance with the invention. Use of such artificial chordae are useful in myriad other cardiac applications as described herein. It will be appreciated by those of skill in the art that the steps of the procedures described herein need not be practiced in the identical order disclosed herein, but may instead be practiced in any suitable order.

In accordance with one example, a prolapsing valve leaflet may first be captured to anchor an artificial chord thereto.

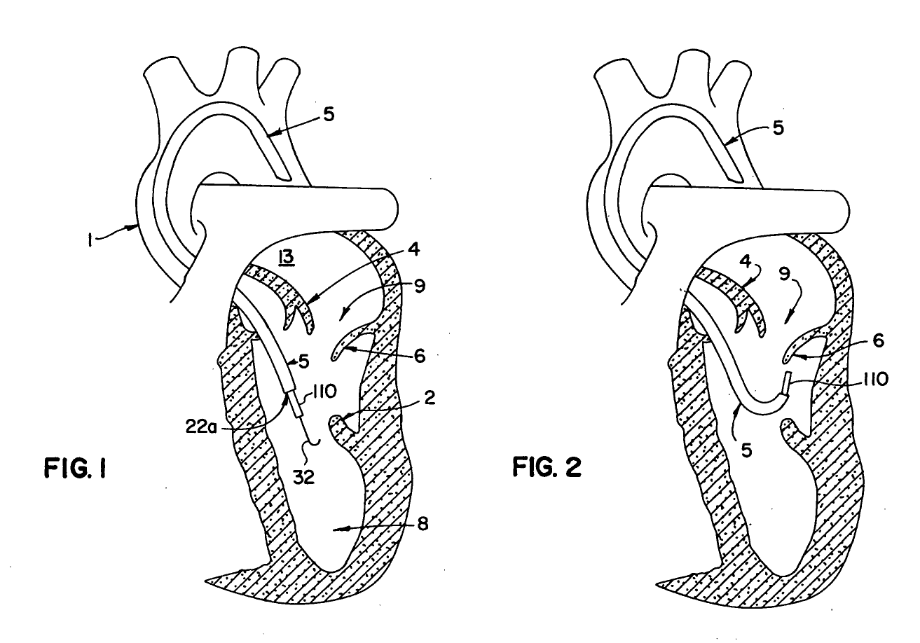

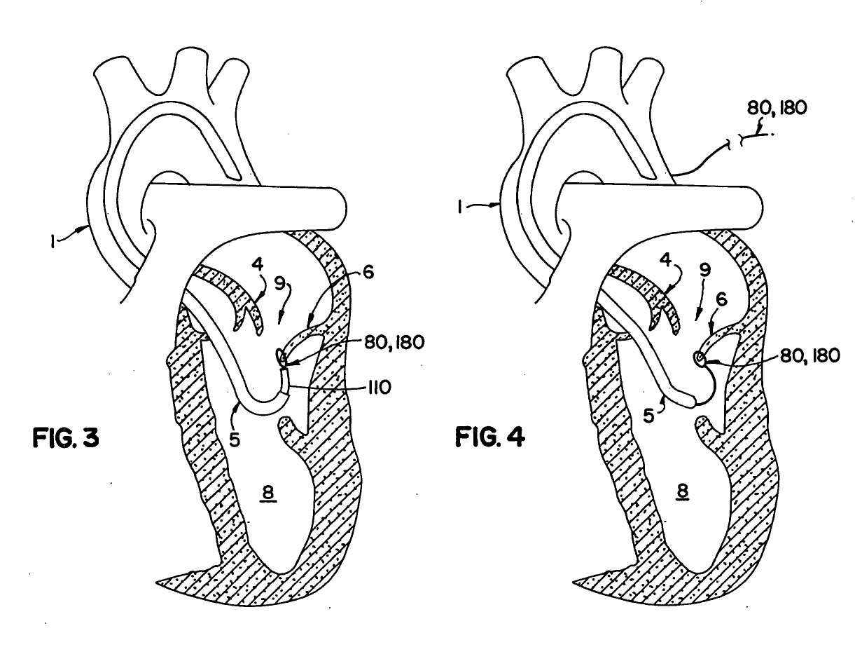

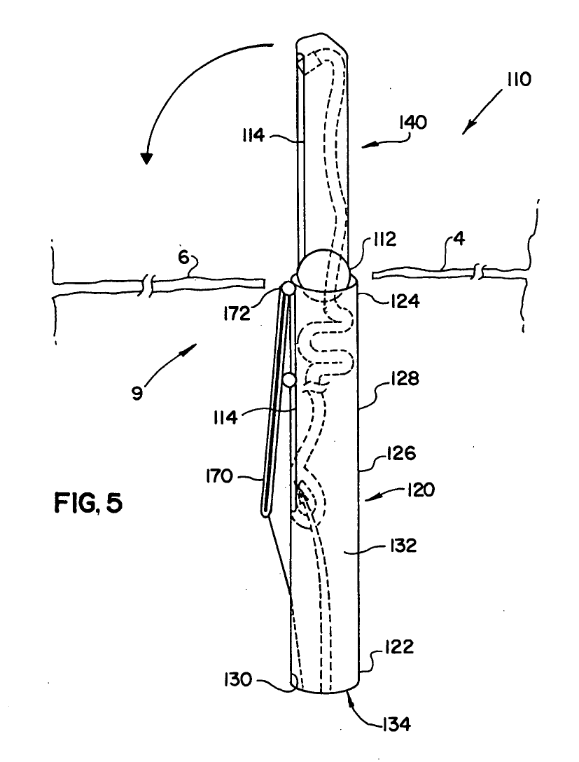

For purposes of illustration, and not limitation, as embodied herein and as depicted in FIG. 1, a guide catheter 5 may be directed through a patient's aorta 1 into the patient's left ventricle 8. As depicted in FIGS. 1 and 2, a leaflet grasping catheter 110 is advanced through guide catheter 5 toward a prolapsing leaflet 6 of a patient's mitral valve 9. Catheter 110 is adapted and configured to pass a filament or artificial chordae, preferably made from suitable suture material, through the prolapsing leaflet 6 as depicted in FIGS. 3 and 4.

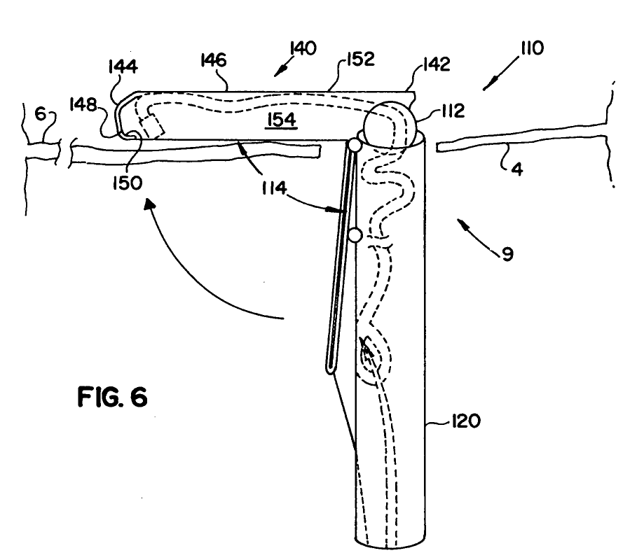

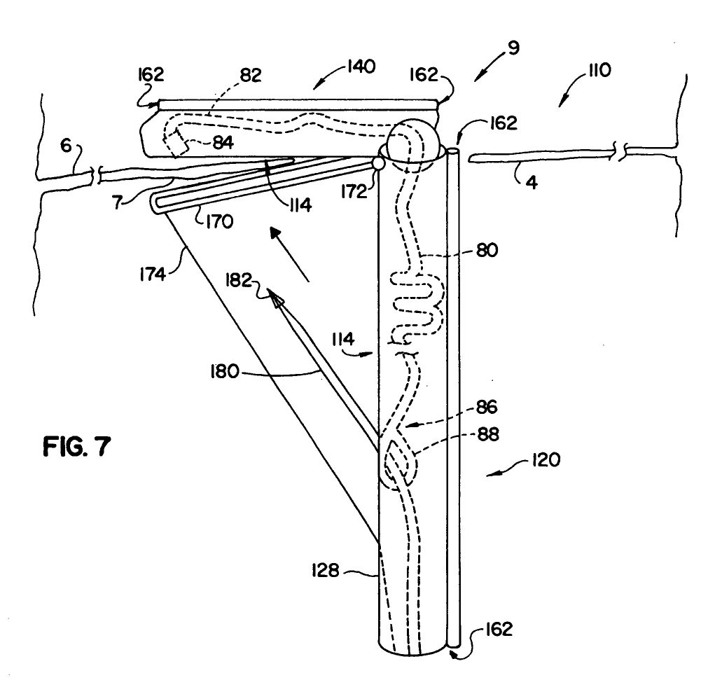

While it may be appreciated that any suitable catheter may be used, a description of one embodiment of an exemplary leaflet catheter 110 is depicted in FIGS. 5-10. As embodied in FIGS. 5-10, leaflet catheter 110 can include a proximal portion 120 connected to a distal portion 140 by way of a rotatable hinge 112. As depicted in FIG. 5, proximal portion 120 includes a proximal end 122, a distal end 124, and an elongate body 126 having a sidewall 128 with an inner surface 130, and outer surface 132 and defining a lumen 134 therethrough. As depicted in FIG. 6, distal portion 140 includes a proximal end 142, a distal end 144, and an elongate body 146 having a sidewall 148 with an inner surface 150, and outer surface 152 and defining a lumen 154 therethrough. Lumen 134 is adapted to be in fluid communication with lumen 154. A continuous elongate slot 114 is defined through sidewalls 128, 148. Catheter 110 also can include a steering mechanism 160 and/or a guidewire lumen 162 traversing the entire length of catheter 110, or merely along distal portion 140 thereof, as with other rapid-exchange type catheters.

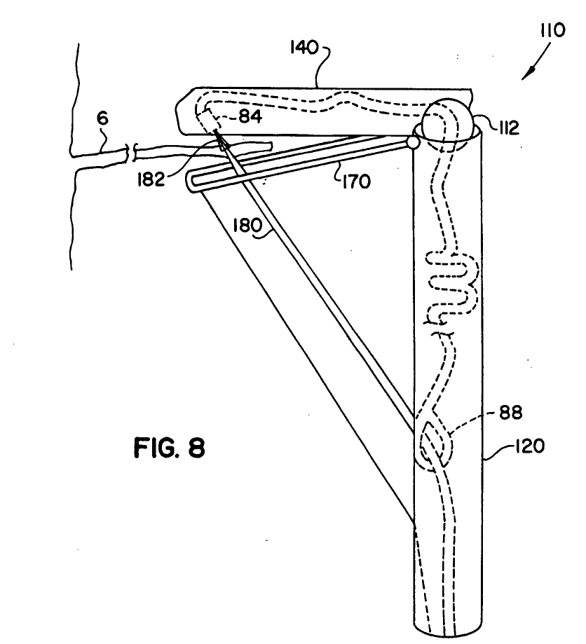

As depicted in FIGS. 1-10, leaflet catheter 110 may be inserted into patient over guidewire 32 and/or through lumen 22 of guide catheter 5. If desired, leaflet catheter 110 could be introduced through a patient's vasculature over a guidewire 32 without a guiding catheter 5. Leaflet catheter 110 is then positioned in a similar manner in the left ventricle 8, but aiming at the mitral valve leaflets 4, 6 as depicted in FIG. 2. Distal portion 140 of leaflet catheter 110 is advanced past the mitral valve leaflets 4, 6 and caused to rotate about ninety degrees such that the portion of slot 114 in distal portion 140 comes into contact with posterior leaflet 6, as shown in FIGS. 5-6. Suction can then be applied through slot 114 to cause leaflet 6 to be drawn toward distal portion 140 and held in position. Additionally or alternatively, as depicted in FIG. 8, a pivotally mounted leaflet retainer 170 can be provided and deployed about pivot 172 using actuator 174 to compress leaflet 6 against distal portion 140. It will be understood that all embodiments depicted herein can be used to perform surgical procedures on any cardiac valve leaflet. For example, while a number of examples described herein refer to posterior leaflet 6, the procedures are equally applicable to anterior leaflet 4.

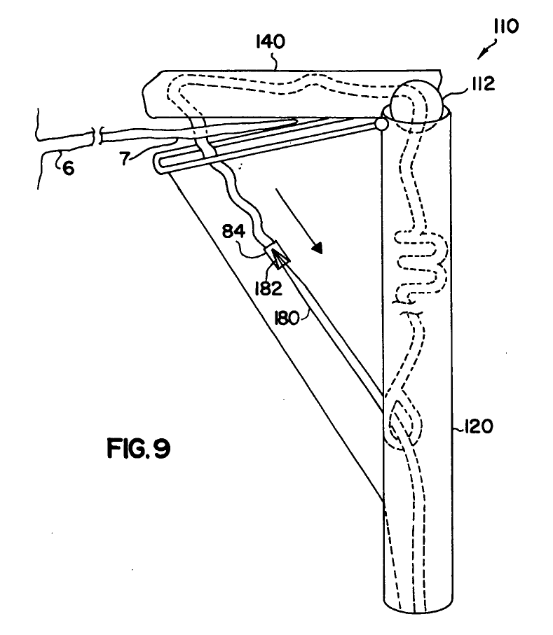

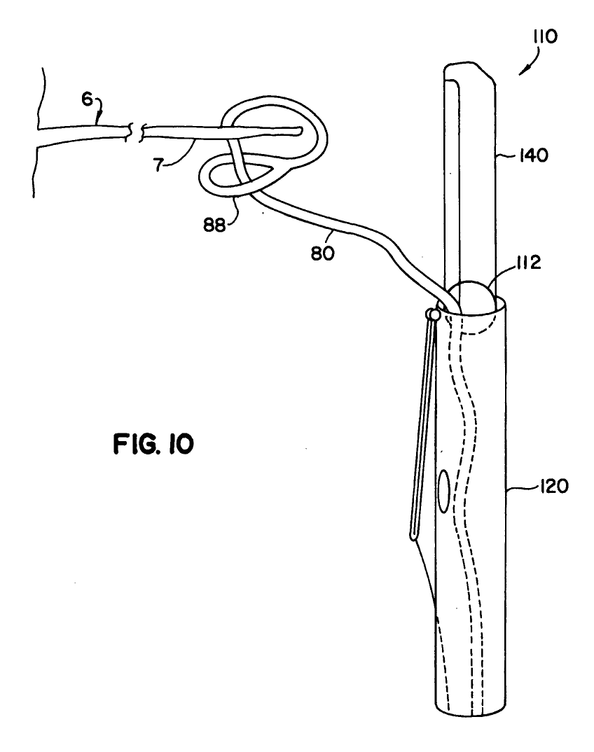

With leaflet 6 held stationary, a deployable needle 180 having a piercing tip 182 can be deployed through wall 128 of proximal section, for example, through slot 114 and through edge 7 of leaflet 6 into engagement with a cuff 84 on first end 82 of suture 80 as illustrated in FIGS. 9-10. Second end 86 of connecting suture 80 includes a loop 88 through which needle 180 is threaded when needle 180 is deployed. Needle 180 can then be retracted proximally as shown in FIG. 9, pulling suture 80 therewith due to the interlocking connection between cuff 84 and tip 182. By virtue of continuous slot 114, suture 80 can then be pulled all the way through as depicted in FIG. 10, forming a knotted loop about edge 7 of leaflet 6.

In addition to direct opposition of the catheter to the leaflet and/or suction applied to the tip of the catheter as described above, other techniques can be used to secure a suture 80 leaflet 6. For example, the system and method of leaflet anchoring may additionally or alternatively include direct suturing, attachment of a clip to the leaflet edge, deployment of pledget material, or deployment of shaped metal such as nitinol through the leaflet. Some of these techniques are described in detail below.

In further accordance with the invention, an artificial chordae affixed to a prolapsing valve leaflet is preferably indirectly or directly affixed to another portion of cardiac tissue.

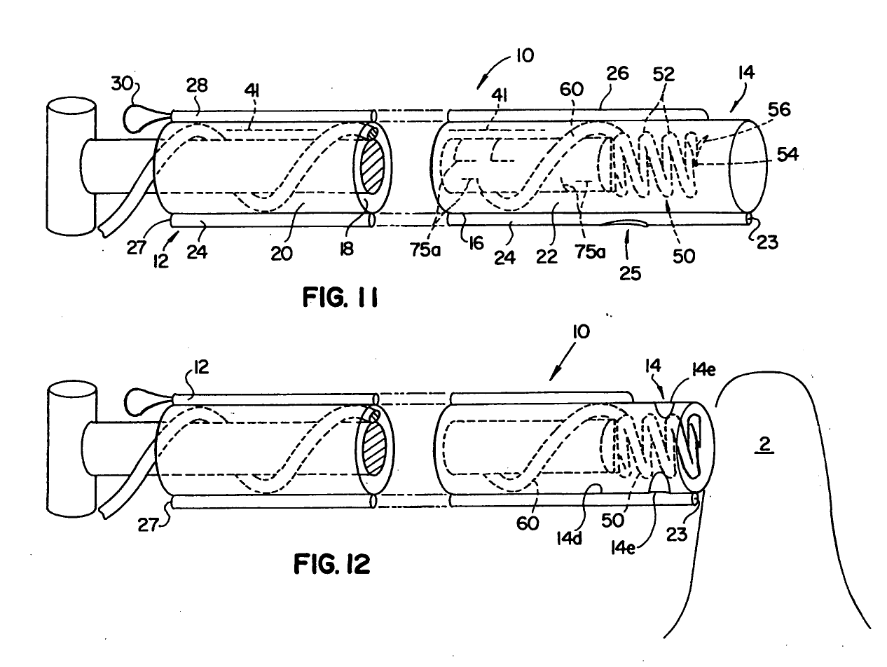

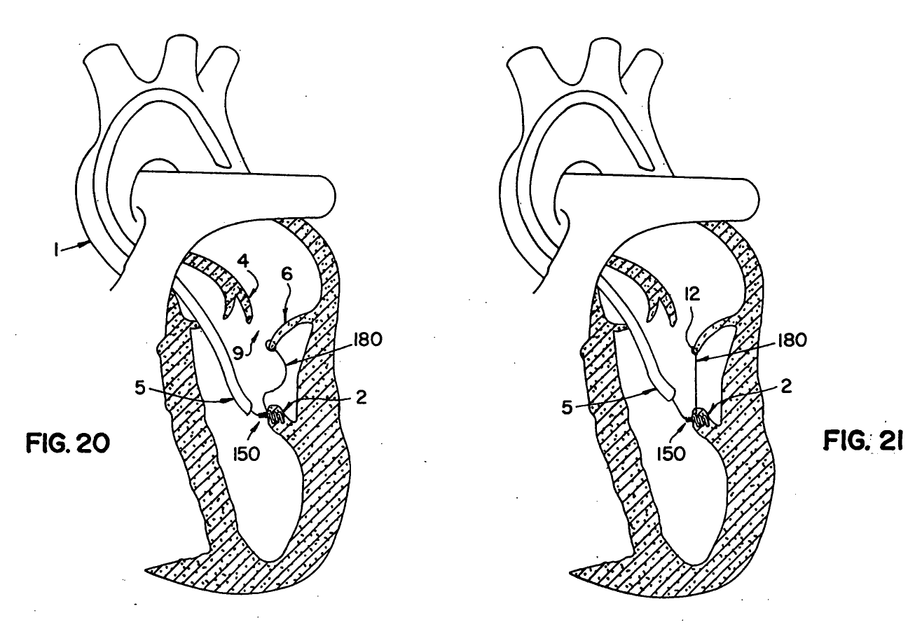

For purposes of illustration and not limitation, as embodied herein and as depicted, for example, in FIGS. 11-19, a first exemplary system and method for affixing artificial chord 80 to cardiac tissue of the patient's heart is presented. In the method and associated system depicted in FIGS. 11-19, chord 80 is affixed to a second chord 60 which, in turn, is affixed to cardiac tissue. In the method and associated systems depicted in FIGS. 20-35, chord 80 is affixed to cardiac tissue without the use of a second chord 60.

While it will be appreciated that many configurations of a suture delivery catheter useful for affixing a suture to cardiac tissue are within the scope of the invention, for purposes of illustration and not limitation, an exemplary embodiment of such a catheter 10 is depicted in FIG. 11. Delivery catheter 10 includes a proximal end 12, a distal end 14 and an elongate body 16 having an inner surface 18, and outer surface 20 and defining a lumen 22 therethrough.

If desired, delivery catheter 10 can be adapted to include a steering mechanism 26 that is operably coupled to an actuator 28 including handle 30 at proximal end 12 of delivery catheter 10. Steering mechanism 26 can be adapted and configured to provide uniplanar or biplanar deflection. Other types of mechanisms that can be used to facilitate steering include use of magnetic guidance, such as internal opposing-pole magnets or use of an external magnetic field for navigation (e.g., Stereotaxis, Inc). Moreover, if a steering system 26 is not provided, as partially depicted in FIG. 16, a guidewire 32 can be provided to traverse the patient's lumenal system prior to introduction of delivery catheter 10. After introduction of guidewire 32, delivery catheter 10 can be introduced by introducing proximal end 34 of guidewire 32 into distal end 14 of catheter 10 and through lumen 18. If desired, a second lumen 24 can be provided in delivery catheter 10 for purposes of introducing over guidewire 32. If a second lumen 24 is provided, second lumen 24 can traverse the entire length of catheter 10 or only a distal portion thereof as with other “rapid-exchange” type catheters. By way of further example, lumen 24 can traverse the entire length of catheter 10 but be provided with one or more intermediate exit ports 25 between distal port 23 and proximal port 27 of lumen 24. Moreover, if a steering system 26 is not provided, the delivery catheter can be introduced through the lumen of a guide catheter with a steering system 26.

Delivery catheter 10 and other catheters described herein can be made from a variety of materials. For example, various polymeric materials may be used, such as nylon and the like. Moreover, it is possible to construct delivery catheter 10 from a multilayer tubular structure incorporating an inner layer of lubricious material, such as HDPE or PTFE and an outer layer of nylon or other comparatively stiff polymeric material. If additional stiffness is required, one or more stiffening wires 41 can be melted into the plastic forming catheter, and/or a layer of braided material, such as stainless steel, can be incorporated be incorporated between successive polymeric layers or melted into or extruded with a single layer of polymeric material. Moreover, a proximal portion of catheter 10 or other catheters described herein can be made at least in part from hypodermic needle tubing “hypotubing” to impart additional stiffness thereto, as desired.

In use, delivery catheter 10 is introduced into the arterial system of a patient e.g., by way of the femoral artery. Delivery catheter 10 can then be advanced through the arterial system and through the aortic valve into the left ventricle 8, for example, as depicted in FIG. 16. Delivery catheter 10 can be steered using uniplanar or biplanar deflection via steering mechanism 26, if provided. Catheter 10 can also be steered by passage through an outer guide catheter or sheath 5 which may include a steering mechanism 26. Catheter 10 can be positioned using image guidance such as echocardiography, adjacent to a papillary muscle head 2 as depicted in FIGS. 12 and 16.

Next, an anchor 50 is provided attached to a length of suture material 60. Anchor 50 with suture 60 attached thereto is anchored into the papillary muscle 2 as depicted in FIGS. 12-16. Anchor 50 includes a helically shaped coil body having a plurality of turns 52 with a distal piercing end 54. A variety of suitable anchors 50 can be used to anchor into the papillary muscle 2. For example, it is also possible to anchor by way of direct suturing, various anchors such as screws, helixes, clips, pins and the like, as described in further detail below. Anchor 50 can be made from a variety of materials such as stainless steel and other metals and composite and/or bioresorbable materials, such as Dacron, Teflon, polypropylene, polytetrafluoroethylene (PTFE), polyvinylchloride (PVC), polydimethylsiloxane, poly(l-lactide), poly(dl-lactide), poly(dl-lactide-co-glycolide), poly(l-lactide-co-dl-lactide), poly(glycolide-co-trimethylene carbonate). Anchor 50 can additionally or alternatively include radiopaque and/or echodense material to facilitate visualization thereof using fluoroscopic or echocardiographic imaging techniques as an aid in implantation. It will be further appreciated by those of skill in the art that anchor 50 need not be helical, but may be formed in any suitable shape for acting as a tissue anchor.

It is also possible to construct anchor 50 from shape memory material, such as nitinol, wherein anchor can be adapted and configured to deploy and expand inside the tissue of papillary muscle 2 to secure suture 60 in place. Moreover, pledget material could be used to facilitate attachment, as described below. Anchor 50 can additionally be provided with one or more barbs 56 (as depicted, for example, in FIG. 11 in a deployed position) to prevent backout of anchor 50 from the papillary muscle 2. If anchor 50 is made from shape memory material such as nitinol, barbs 56 can be adapted and configured to deploy when they reach body temperature. If anchor 50 needs to be removed from the patient after installation, it is possible to locally cool anchor 50 with a cooling device to cause barbs 56 to retract. This can accordingly reduce tissue damage in the event of removal of anchor 50.

Sutures 60, 80 are preferably nonabsorbable, permanent and made from a material that is not likely to cause formation of thrombi thereon. Suitable materials for suture 60 can include, for example, expanded polytetrafluoroethylene (“ePTFE”), obtainable, for example, from W.L. Gore & Associates (Newark, Del.). Preferably, the node and fibril dimensions of the ePTFE suture material will be of suitable dimensions to permit tissue ingrowth therein, such as those described in U.S. Pat. No. 6,436,135 to Goldfarb, the disclosure of which is incorporated herein by reference in its entirety. Other materials can also be used to form sutures herein, either monofilament or polyfilament braided, including polypropylene, polyester, nylon, and silk. Suture 60 can additionally or alternatively include radiopaque and/or echodense properties to facilitate visualization thereof using fluoroscopic or echocardiographic imaging techniques as an aid in implantation.

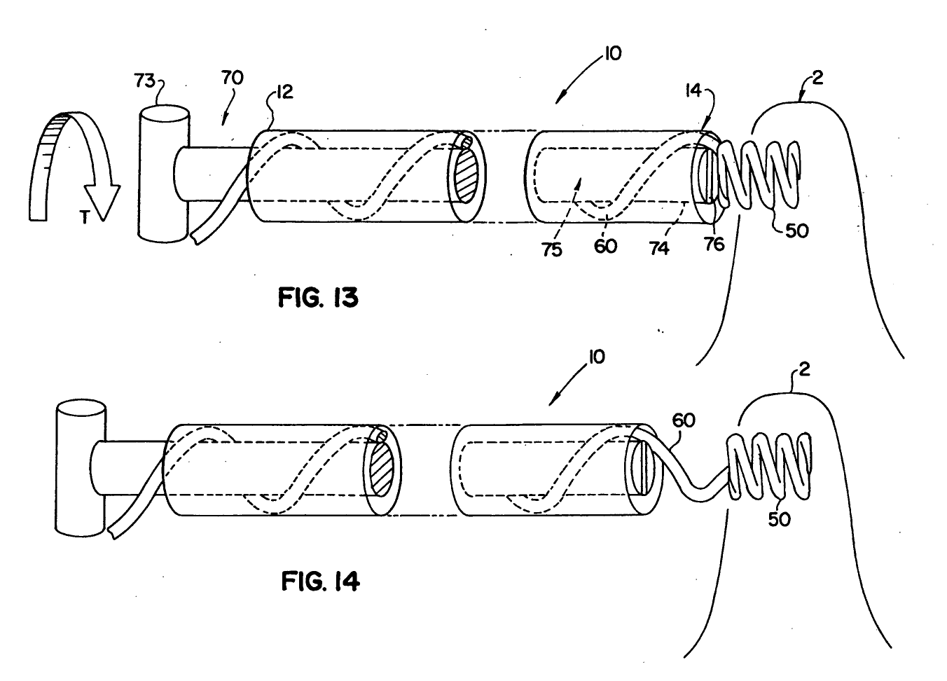

Anchor 50 and suture 60 can be passed through lumen 22 of delivery catheter 10 using an inner catheter 70 disposed inside lumen 22 as depicted in detail, for example, in FIG. 13. Inner catheter 70 includes a proximal end 72 having a handle 73, a distal end 74 and an elongate body 75. Inner catheter 70 further includes an anchor engaging portion 76 (e.g., a groove or the like) to engage with anchor 50. As depicted in FIG. 13, fitting 76 includes a groove that is adapted and configured to engage an engagement portion 58 of anchor 50. By applying a torque T to handle 73, anchor 50 is screwed into papillary muscle 2. Additionally, the distal tip 14 of catheter 10 may incorporate one or more guides 14(e) on the inner surface 14(d) to facilitate appropriate helical/forward motion of anchor 50. This may include, for example, a spiral groove to accommodate helical anchor and/or may include a series of spaced protruding members or knobs 14(e) on the inner surface of catheter 10 to guide the forward movement of anchor 50. Providing guides 14(e) can be advantageous as it helps ensure that anchor 50 will not be pushed out of the end of catheter 10 without sufficiently implanting it, and also ensures that anchor 50 is advanced distally as it is rotated at an appropriate rate (e.g., the pitch of the threads of anchor 50) to prevent maceration of tissue and helping to ensure a successful implantation.

Elongate body 75 may include a plurality of cuts therein to provide regions of varying flexibility. For example, it by be desirable for a distal region of elongate body 75 to be more flexible than a proximal region thereof. To accomplish this, a plurality of longitudinal and or radial slots 75a may be disposed therein. Suitable configurations of slots 75a are described, for example, in U.S. Pat. No. 5,477,856, which is incorporated by reference herein in its entirety. By way of further example, varying stiffness of any portion of any catheter described herein can be supplied in accordance with the teachings disclosed in U.S. Pat. Nos. 5,399,164, 5,605,543, and 5,674,208, for example. Each of these patents is hereby incorporated by reference herein in its entirety.

After anchor 50 is secured in papillary muscle 2, inner catheter 70 can be withdrawn from the patient. As depicted, after removal of inner catheter 70 and leaflet catheter 110, sutures 60 and 80 traverse the entirety of lumen 22 to a point outside of the patient. As a variation, it is possible to coil suture about distal end 74 of inner catheter 70 such that it uncoils as catheter 70 is withdrawn proximally through lumen 22 and out of catheter 10. If desired, delivery catheter 10 can next be withdrawn from the patient, leaving a trail of suture material 60 through the vascular system and outside of the patient as depicted in FIG. 5. While catheters 10, 110 have been depicted herein as discrete devices, it is within the scope of the invention to have all functions performed by these catheters to be performed by a single catheter to facilitate the procedure.

Next, as depicted in FIGS. 17-19, a fastening catheter 210 may be introduced through guide catheter 5 to the operating site in order to facilitate attachment of sutures 60, 80 to each other to form an artificial chordae. As depicted in FIG. 17, fastening catheter 210 includes a proximal end 215, a distal region 214 and an elongate body 216 that can include a lumen 218 therethrough. Distal region 214 of catheter 210 can also be provided with a device 220 disposed proximate distal tip 212 for attaching the sutures 60, 80 to each other to form an artificial valve chord, described in detail below. In operation, fastening catheter 210 can be introduced over or along side of sutures 60, 80 and positioned in the left ventricle 8.

If delivery catheter 110 or other guiding catheter is not used and sutures 60, 80 are exposed to the patient's bloodstream, catheter 210 can be introduced alongside sutures 60, 80 if, for example, catheter 210 is provided with a steering mechanism as disclosed herein or inserted over guidewire 32. Catheter 210 can alternatively be introduced over guidewire 32 by inserting guidewire 32 through lumen 218. Lumen 218 can traverse the entirety of the length of catheter 210 or a portion thereof. By way of further example, if lumen 218 traverses the entirety of catheter 210 it can be provided with one or more exit ports 219 located proximate distal end 214 of catheter 210. Alternatively, catheter 210 can be introduced over one or more of sutures 60, 80 by threading sutures through lumen 218 and introducing catheter over sutures. In this embodiment, it can be advantageous to use an exit port proximate the distal end of the catheter to facilitate introduction.

Alternatively, it may be desired to leave delivery catheter 10 (or other guiding catheter) in place so that sutures 60, 80 traverse the length of lumen 22. Catheter can accordingly be introduced either independently of sutures 60, 80, or over sutures 60, 80 as described above.

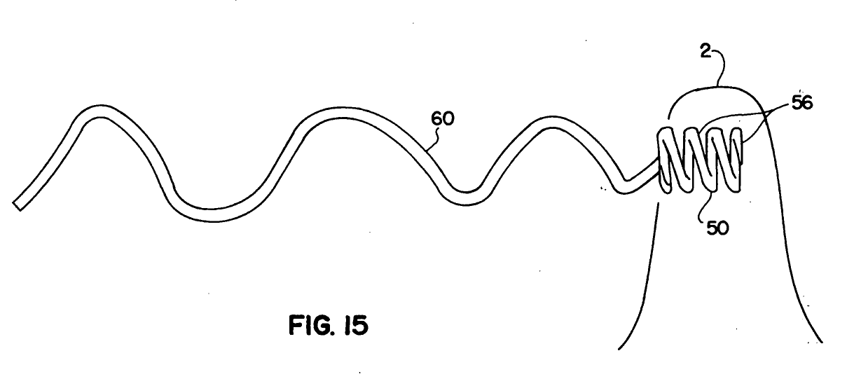

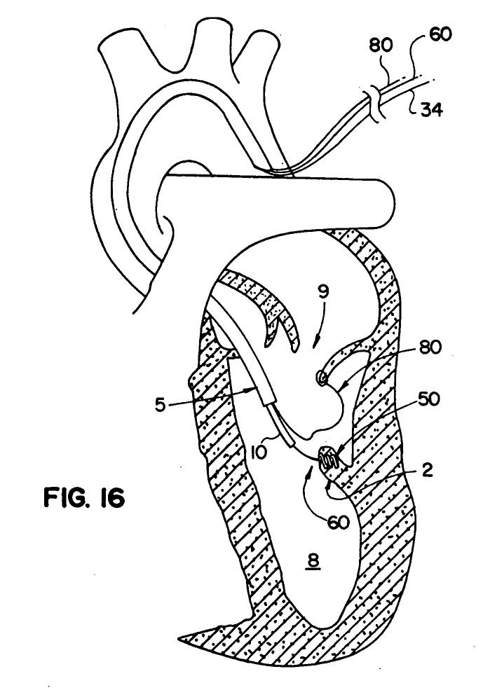

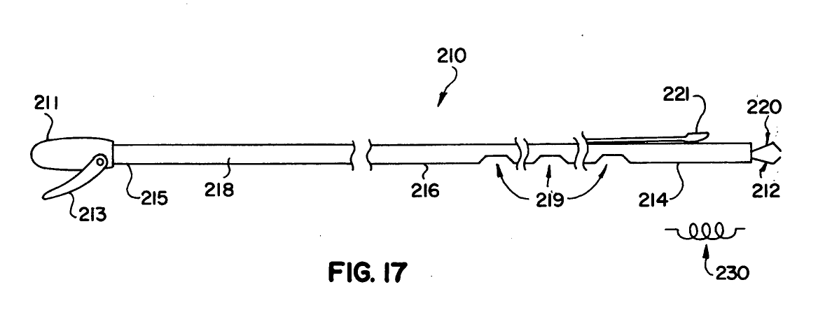

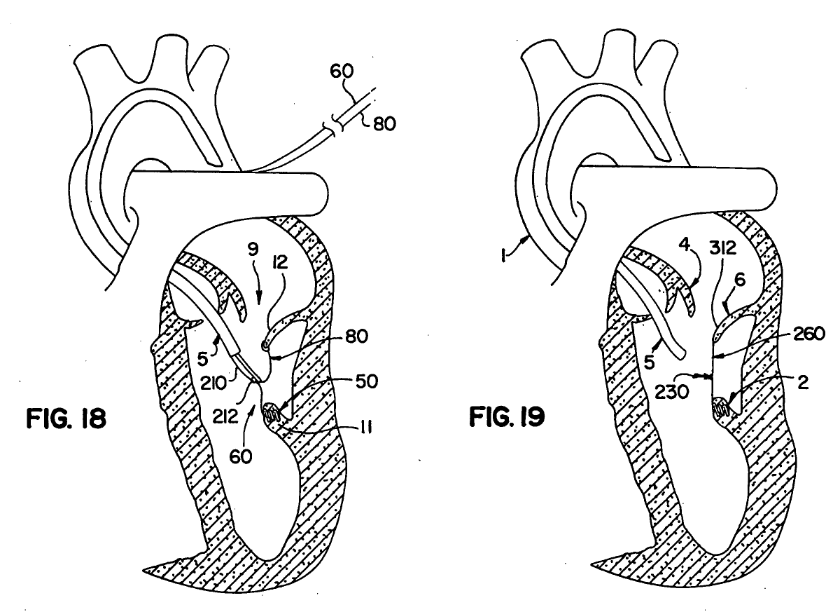

Once the proximal ends 61, 81 of sutures 60, 80 have been exteriorized from the patient, they can be manipulated by the operator as depicted in FIG. 18. Next, the distal tip 212 of catheter 210 is positioned below the mitral valve 9 between the two previously placed anchor points of sutures 60, 80. Tension is applied to each of the sutures 60, 80 by the operator such that they are coupled proximate the distal end 212 of catheter 210. Sutures 60, 80 can be brought into proximity by virtue of threading them through lumen 218 or lumen 22, as desired. At this point, sutures 60, 80 effectively form a single length of suture running from anchor point 311 on the papillary muscle head to the anchor point 12 on the mitral valve leaflet 6, as depicted in FIG. 19. The length of this combined artificial chord 260 can be adjusted by altering the amount of suture material 60 from papillary muscle 2 to catheter 210 and/or the amount of material from leaflet 6 to catheter 210. Preferably, this procedure is performed under image guidance such as echocardiography in order to be certain that the length of chord 260 is correct. The operator can determine the correct length of chord 260 by experimentation. That is to say, the length of suture 260 can be adjusted until the operator is satisfied that artificial chord 260 is at a suitable length to reduce, and preferably minimize, mitral valve regurgitation.

Once the desired length of suture 260 is achieved, the two sutures 60, 80 are affixed to one another by fastening device 220 located at distal end 214 of catheter 210 as shown in FIG. 19. Fastening device 220 can accomplish this function in a variety of ways. For purposes of illustration and not limitation, as depicted in FIGS. 17 and 19, a clip or fastener 230 can be deployed by fastening device 220. Fastening device 220 can be actuated by an actuator 213 mounted on a handle 211 disposed at the proximal end 215 of catheter 210. The tails of sutures 60, 80 are cut by a cutting mechanism 221 disposed on fastening device 221 and removed, then catheter 210 is removed. The patient is now left with a single artificial chord created by the union of two separately placed sutures. Additional artificial chordae 260 may be placed, as dictated by the clinical situation. More generally, fasteners 230 as depicted herein may include any suitable clip or suture that can mechanically compress two or more sutures together without slipping once the clip is attached. Alternatively, the chordal sutures can be permanently joined by thermal or chemical means.

By way of further example, still further embodiments of systems and methods in accordance with the present invention are provided.

With continued reference to FIGS. 1-4, FIG. 1 illustrates the left-sided structures of the heart, including aorta 1, left ventricle 8, papillary muscle 2, left atrium 13, and mitral valve 9 including posterior mitral leaflet 6, and anterior mitral leaflet 4. A guide catheter 5 is shown in the aorta 1, traversing the aortic valve to enter the left ventricle 8. Preferably, guide catheter 5 will be deflectable to allow for steering and positioning, although it is within the scope of the present invention to use multiple guide catheters of fixed or variable shapes could also be used to position the described devices. The guide catheter 5 is shown positioned in the left ventricle 8 in order to achieve a repair of the mitral valve 9.

As depicted, the guide catheter 5, once in the ventricular cavity, is directed toward the atrioventricular (mitral or tricuspid) valve leaflet edge using image guidance, as illustrated in FIG. 2. This image guidance may include, but is not limited to, echocardiography, fluoroscopy, computed tomography, magnetic resonance imaging, and intracardiac imaging utilizing technology such as catheter-based infrared imaging. A leaflet catheter (e.g., 110) is passed through the guide catheter 5 and the prolapsing valve leaflet segment 6 is engaged by the catheter and a nonabsorbable suture 180 is anchored to the leaflet edge as shown in FIGS. 3-4. As embodied herein, the leaflet 6 may be engaged by one of several methods including direct opposition of the catheter to the leaflet, suction applied to the tip of the catheter, a snare positioned on the atrial side of the leaflet 6, or a cross-bar to catch the underside of the leaflet, and the like. The system of leaflet anchoring may include but is not limited to direct suture, attachment of a clip to the leaflet edge, deployment of pledget material, or deployment of shaped metal such as nitinol through the leaflet. As described herein, suture 180 is formed from a permanent, flexible and relatively inelastic biocompatible material such as ePTFE but other suitable materials that may be used as described herein. Multiple such sutures may be placed as deemed appropriate to achieve repair of the dysfunctional valve 9.

Next, the leaflet catheter is withdrawn through the guide catheter 5 such that the suture 180 affixed to the leaflet is traverses the length of the guide catheter 5 and is exteriorized as depicted in FIG. 4. If multiple guide catheters are to be used, the guide catheter can be removed from the patient, leaving the proximal end of the suture exteriorized through the arterial access sheath. Subsequent catheters may then be passed either over or alongside the existing suture for the remainder of the procedure.

As depicted in FIG. 20 a second delivery catheter (such as catheter 70 depicted herein) can be passed through guide catheter 5 and positioned toward one of the papillary muscles 2. The correct positioning and appropriate papillary muscle 2 is directed using the image guidance as described for the leaflet capture. The delivery catheter places a papillary muscle anchor 150 into the papillary muscle head 2. In the presently preferred embodiment, the papillary muscle anchor 150 has a permanently attached suture fastener through which the previously placed suture 180 is passed as depicted in FIG. 21. This can be loaded upon initially passing the second delivery catheter into the patient. If multiple sutures 180 had been placed into the valve leaflet, these may all be passed through the fastener 150 of a single papillary muscle anchor or may be passed individually through separate anchors 150, depending on the anatomic situation.

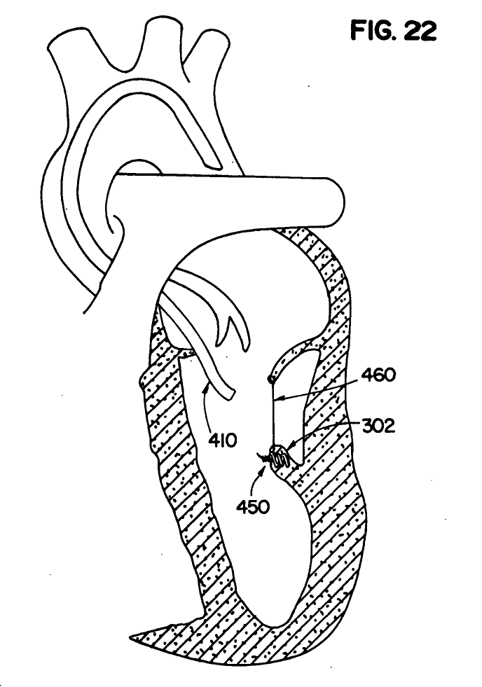

Next, as depicted in FIG. 21, the length of the artificial chorda(e), can be adjusted by applying tension to a the suture 180, which was previously exteriorized through the guide catheter or arterial access sheath 5. The fastener 150 acts as a grommet, or fulcrum, such that the artificial chorda(e)/suture 180 now forms a straight line from papillary muscle anchor 150 to the leaflet attachment site 12. The length of the artificial chorda(e) can be adjusted under the image guidance previously described by altering the amount of applied tension to the end of the suture. Once the appropriate length of suture is reached to achieve the desired clinical effect, the fastener 150 is secured to fix the length of the suture/artificial chorda(e) 180, the suture 180 is cut proximate to the fastener 150, and the guiding catheter 5 is removed, leaving a fixed length artificial chord 180, as depicted in FIG. 22.

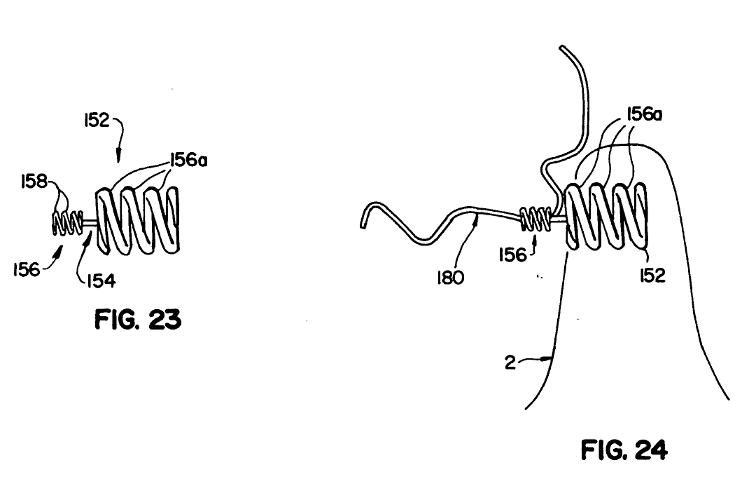

As depicted in FIGS. 23-24, a first embodiment of anchor member 150 includes a first or anchor portion 152 for anchoring into papillary muscle head 2 coupled to a suture lock 156 by way of a connecting portion 154. As depicted, the suture 180 previously passed through leaflet 4 can be passed through the lumen lock portion 156 of anchor 150 before anchor 150 is passed into the patient through the guide catheter, thus facilitating chordal length adjustment. As will be appreciated, anchor 150 can take on a variety of forms conducive to this type of procedure. After an appropriate length has been determined for the suture 180, the lock portion 156 can be crimped or otherwise caused to bear down on suture, locking it in place. This and other embodiments of lock 156 are described in detail more fully below.

By way of further example, anchor 150 can be formed of a shape memory material such as nitinol. Anchor 150 can be fixed in a larger diameter configuration until deployed, then assume a resting state of a smaller inner diameter sufficient to lock the suture(s) in place, for example, between turns 158 of coil making up guide portion 156, or by guide portion contracting and bearing down on suture 180. If desired, one or more barbs 156(a) can be provided to prevent the back out of anchor. If formed of shape memory material, for example, barbs 156(a) may be adapted and configured to deploy after implantation as described herein.

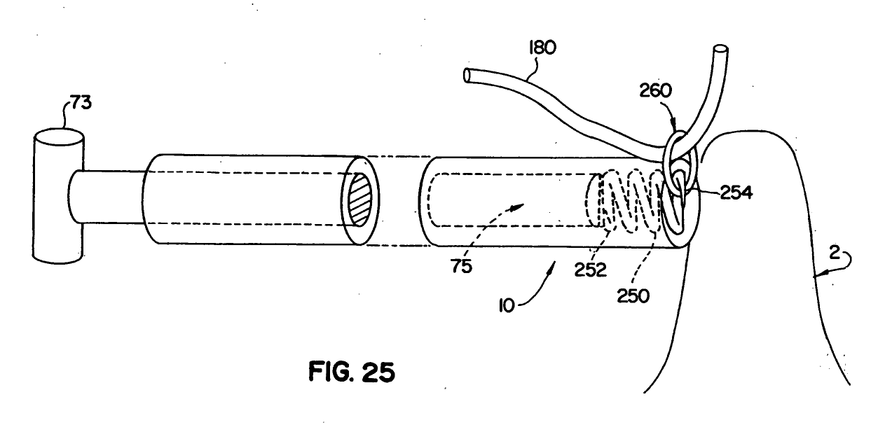

By way of further example, an additional embodiment of an anchor 250 and an associated method of implantation are illustrated in FIGS. 25-27. As depicted in FIGS. 25-27, suture 180 is not attached to the anchor 250 directly, but uses a coupling 260 operatively associated with anchor 250 whereby the suture 180 extending from the leaflet 6 which has been previously exteriorized from the body is passed through this coupling 260 that is attached to the anchor 250. Thus, once the anchor 250 is deployed by way of catheter 10, the coupling 260 acts as a guide or fulcrum whereby pulling on the suture 180 outside the body urges catheter 10 and/or guide catheter 5 against the papillary muscle 2 to facilitate adjusting the length of suture/artificial chord 180 from anchor 250 to leaflet 6. FIG. 25 depicts the anchor delivery catheter 10 (which, as will be understood by those of skill in the art, can be passed through the same deflectable guide catheter or sheath 5 used to pass the leaflet delivery catheter 110) containing anchor 250, actuating rod 75 affixed to torquable handle 73 and a coupling member 260. The coupling member 260 can be a simple ring or figure-eight ring, or other suitable shape, that is temporarily affixed to the end of catheter 10. The coupler 260 accommodates both the tip of the anchor 250 and the suture 180 from the leaflet 6.

As seen in FIG. 26, as the anchor 250 is rotated and advances through the papillary muscle tissue, the coupler 260 stays fixed relative to catheter 10 and winds down the anchor 250 from its distal tip 254 to its proximal end 252. The proximal end 252 of the anchor 250 is closed upon itself to prevent the coupler 260 from coming off the end. Thus, as depicted in FIG. 26, as the anchor 250 is driven into the papillary 2, the coupler 260 stays on the outside of the papillary 2. Moreover, by staying in a fixed position as the anchor 250 rotates, the suture 180 is prevented from becoming entangled by the rotating mechanism of the anchor 250. The suture 180 can then be freely adjusted in length until the operator is ready to fix the suture in position by a locker as described herein, or other suitable method. Once the anchor 250 is fully deployed in to the papillary 2, the entire assembly, including the coupler 260, may be released from the delivery catheter as shown in FIG. 27.

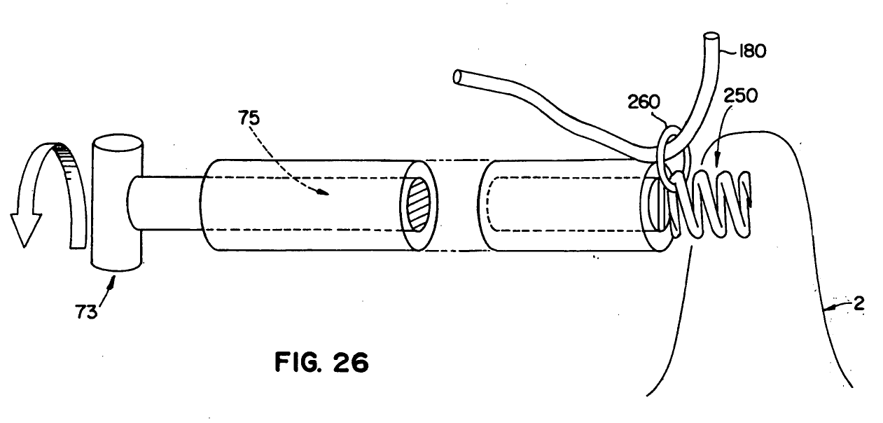

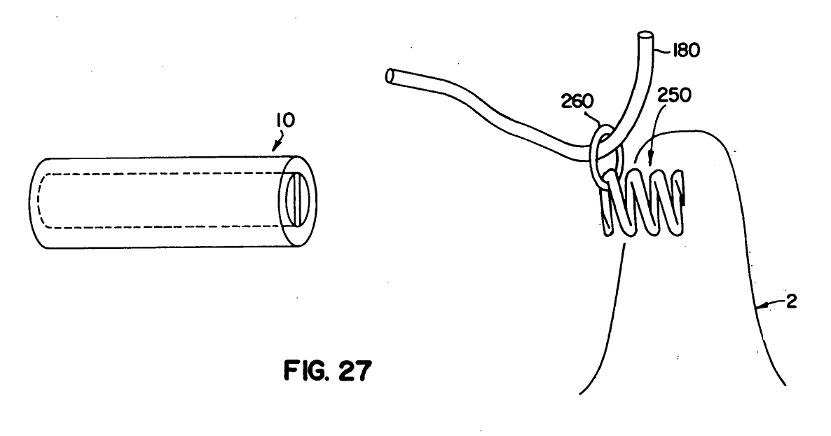

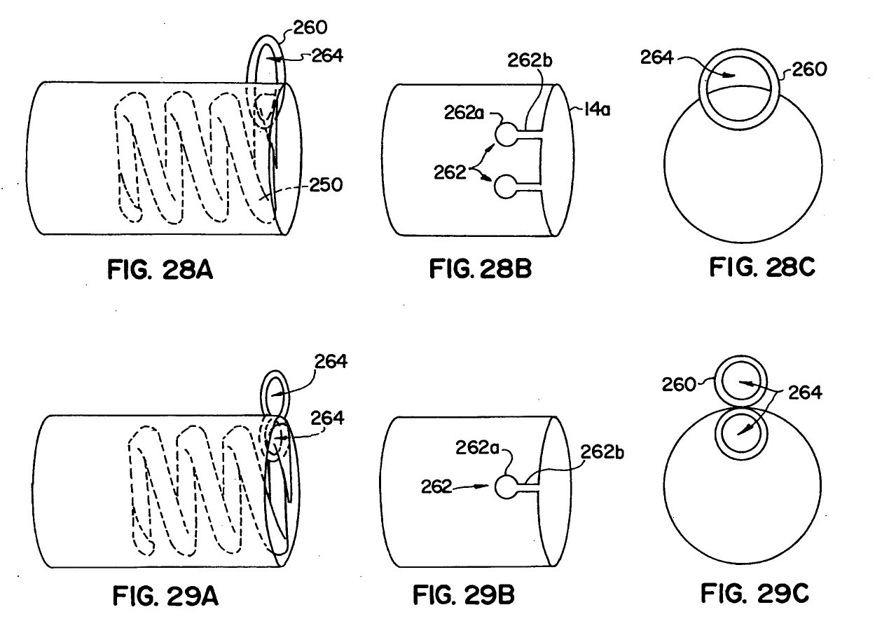

FIGS. 28-29 depict further embodiments illustrating different methods of temporarily fixing the anchor coupler 260 in the tip of the delivery catheter 10. The distal tip portion 14 of delivery catheter 10 can be a short segment of hypotube affixed to delivery catheter 10 or simply be made from a plastic and/or composite material (e.g., carbon fiber reinforced material) having stiffer properties than the certain other portions of the catheter 10. For example, if made of nylon, delivery catheter can include a distal tip portion made of stiffer nylon than more proximal segments of the delivery catheter 10. FIGS. 28(a)-28(c) depict the coupler 260 as a simple ring, with one half inside the distal region 14 of the delivery catheter 10 and one half outside thereof. FIG. 28(a) represents a schematic side view of the distal region 14 of the delivery catheter, FIG. 28(b) represents a top view of the distal region 14 without the coupler 260 in place, and FIG. 28(c) depicts an end view of the distal region of the catheter 10 with the coupler 260 in place. FIG. 28(b) clearly indicates the distal region 14 having two cutout slots 262, each slot 262 being defined by a rounded coupler receiving portion 262(a) and a slightly narrower groove portion 262(b) connecting the coupler receiving portion to the extreme distal tip 14(a) of catheter 10. In operation, coupler 260 is securely held in place by coupler receiving portions 262(a) while the anchor 250 is still in the catheter 10, but can be pushed out through groove portions 262(b) by deflecting the material from which distal region 14 is formed once the anchor has been deployed.

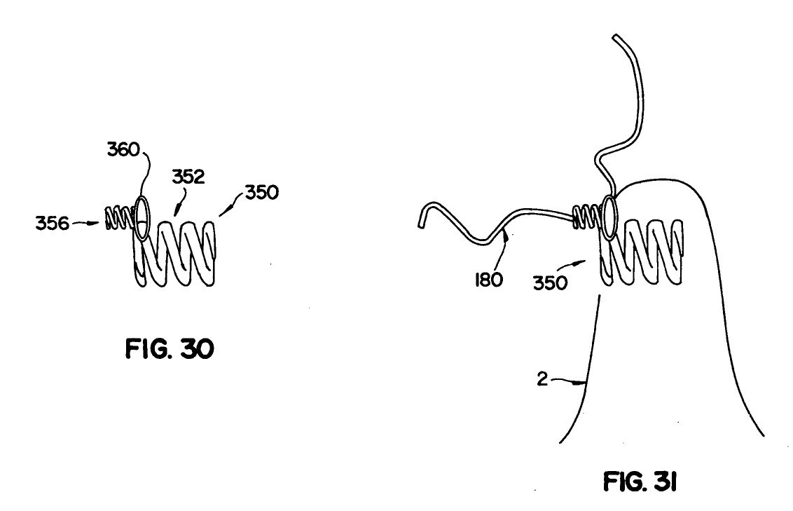

The anchor 250 is preferably preloaded in the delivery catheter 10 such that the tip of anchor 250 is passed through the a lumen 264 of the coupler 260, ensuring that forward rotation of the anchor maintains the coupler on the anchor. FIGS. 29(a)-29(c) show a similar set of schematics wherein the coupler 260 is of a figure-eight shape rather than a simple ring, with one lumen 264 for the anchor 250 and one for the suture 180. By using one of these couplers 260 in combination with a suture locking mechanism (described in detail below) that is larger in diameter than the lumen 264 of the coupler 260 can effectively secure the length of the suture/artificial chord 180. FIGS. 30-31 depict still further embodiments of anchor 350. Anchor 350 is similar to the anchor 150 depicted in FIGS. 23-24, except that anchor 350 further includes coupler 360 and lock 356. Lock 356 is similar to lock 156 described above, and to the locks described in detail below.

In further accordance with the invention, a suture locking system is provided to lock a suture into place to maintain tension on an artificial chord(ae).

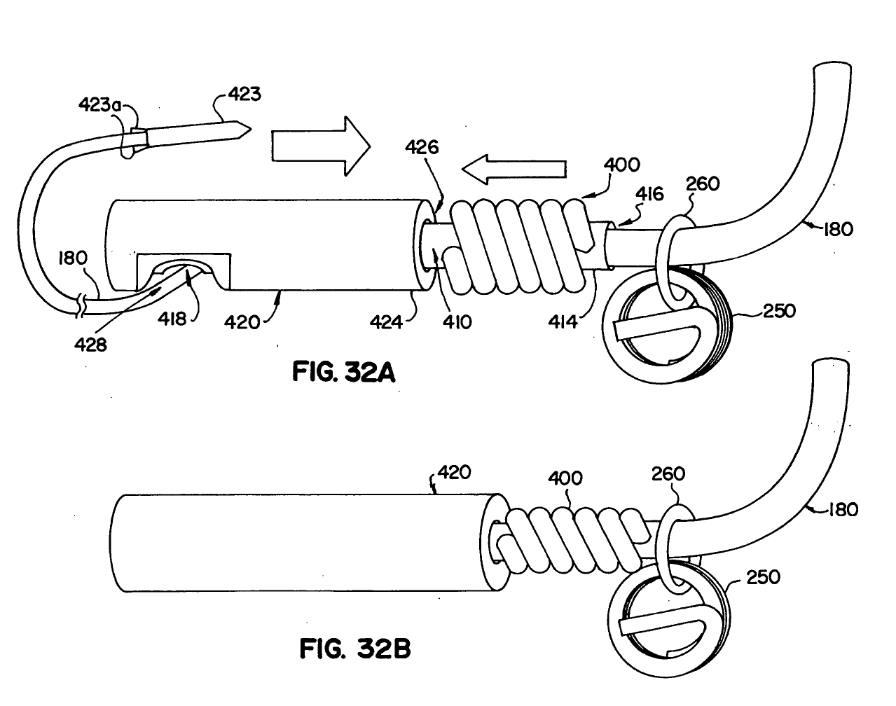

For purposes of illustration and not limitation, as embodied herein and as depicted in FIGS. 32-35, a variety of suture lock or retainers 400 and supporting devices are depicted. As depicted in FIGS. 32(a)-32(b), a first representative embodiment of a suture lock or retainer 400 is depicted. The suture 180 is seen passing through the coupler 260 on the anchor 250. Suture 180 is further directed through an inner catheter 410, preferably having a rigid distal tip 414 as described herein. Inner catheter 410 is accordingly slidably disposed within a lumen of an outer catheter 420. As is further depicted, a lock 400 is provided that is adapted and configured to radially contract from a first, larger diameter condition to a second, smaller diameter condition. As depicted, lock 400 is maintained in the first condition by virtue of lock 400 being wound around inner catheter 410, which has an outside diameter that is larger than that of suture, since suture passes through a lumen defined by inner catheter 410. As inner catheter 410 is withdrawn proximally into the distal end 424 and interior of the lumen 426 of outer catheter 420, lock 400 is pushed off of inner catheter 410 and permitted contract into its second, smaller diameter condition about suture 180, thereby clamping itself about suture 180. Preferably, the lumen 426 of outer catheter 420 is substantially smaller than the outer diameter of lock 400 when lock 400 is in its second condition. As depicted, lock 400 is a coil but could include of a number of different suitable shapes and materials—preferably shape memory material such as nitinol that will assume a fixed shape when pushed off of the inner catheter 410. Rapid exchange ports 418, 428 can be provided in each of inner catheter 410 and outer catheter 420, respectively, a substantially large distance from the proximal ends 412, 422 of catheters, but a substantially short distance (e.g., about 5, 10, 15, 20, 25 or 30 cm.) from the distal ends 414, 424 of catheters 410, 420 to permit ready introduction of catheters 410, 420 over suture 180 to facilitate applying a lock 400 to suture 180. If desired, suture 180 may include or be attached to a relatively stiff elongate member (e.g., stylet 423 via clips 423(a)) to facilitate passage of suture 180 through the lumen of any catheter embodied herein. Catheters 410, 420 can additionally include a suture severing mechanism for severing suture 180 when a procedure is complete. Exemplary embodiments of severing mechanism are described in detail below.

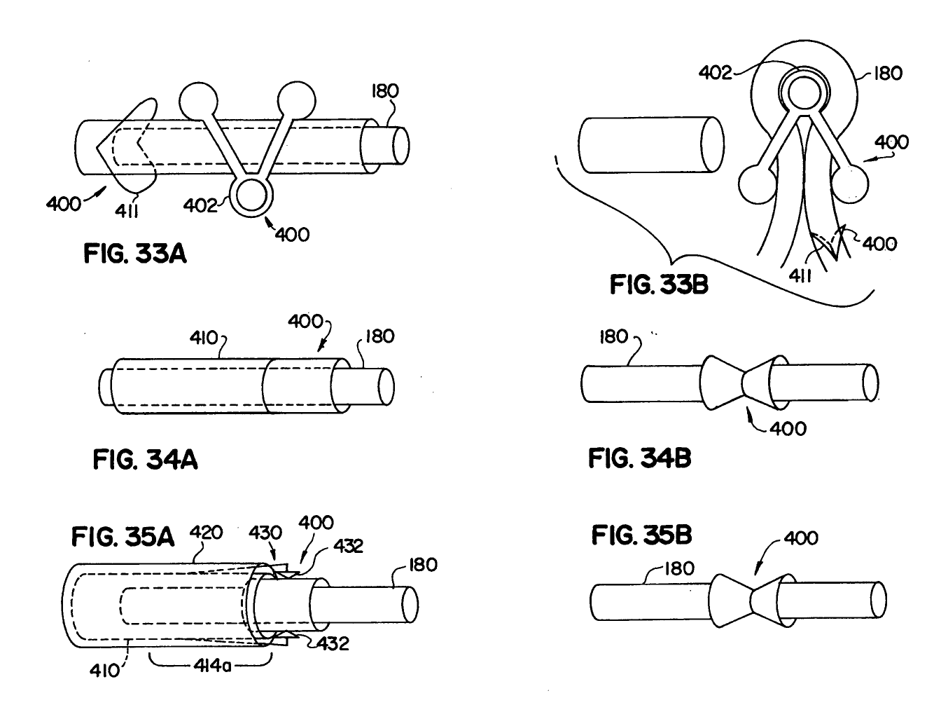

FIGS. 33-35 depict further exemplary locking mechanisms 400. The embodiment of FIG. 33 uses a constraining inner catheter 410 as with the embodiment depicted in FIG. 32, but additionally uses a buckle and spring mechanism. The spring 402 (represented by the open circle) is forced into an open position by virtue of the leg portions 404 of lock 400 being splayed apart by surrounding the rigid inner catheter (made from a stiff material, e.g., hypotube) through which the suture 180 passes. As the inner catheter 410 is withdrawn into outer catheter 420, the spring 402 assumes its resting position, trapping the suture 180 and locking it in place.

A second filament lock 400 is depicted in FIG. 33, comprising a pair of jaws 411 that are biased toward each other. When deployed, jaws 411 clamp down on suture 180.

The embodiment depicted in FIG. 34 utilizes a rigid inner catheter 410 (e.g., containing a hypotube), but uses a tube 400 of nitinol or similar shape memory material that is held open by the inner catheter 410, but when released from the inner catheter 410 assumes its resting position, which in turn crimps the suture 410.

By way of further example, the embodiment depicted in FIG. 35 includes an active crimping mechanism that plastically deforms lock 400 about suture 180. For example, lock may be pushed into place by the combination of an inner catheter 410 and an outer catheter 420. However, lock 400 does not ride on inner catheter 410. A crimping mechanism 430 is disposed about and in operable association with the distal region 414(a) of inner catheter, wherein jaws 432 of crimping mechanism 430 bear down on lock 400, for example, when outer catheter 420 is slid over crimping mechanism 430.

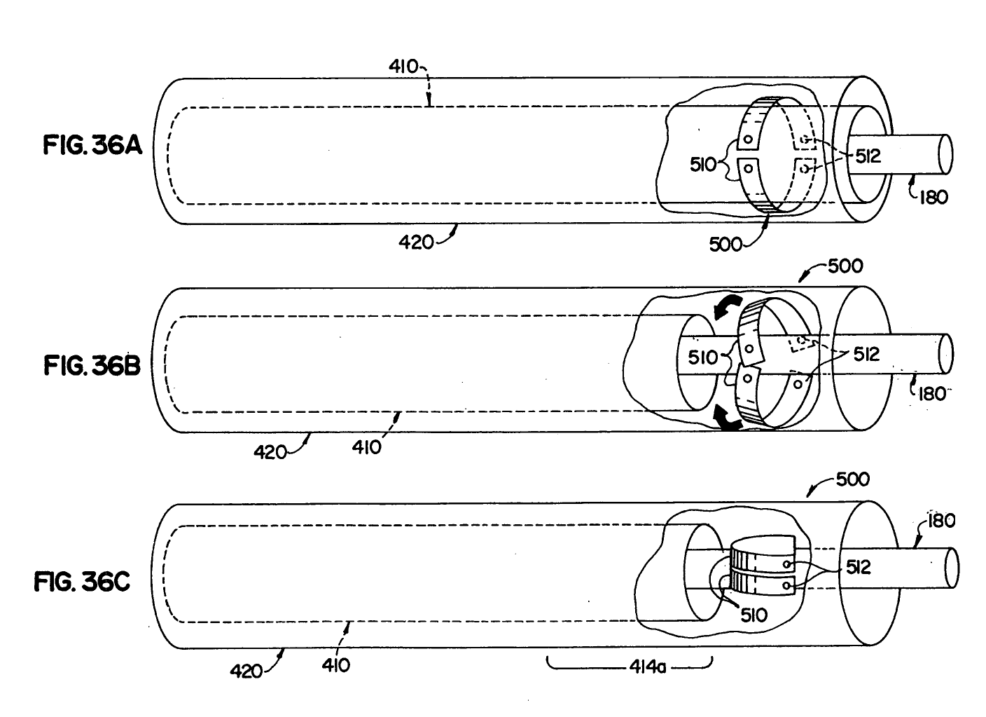

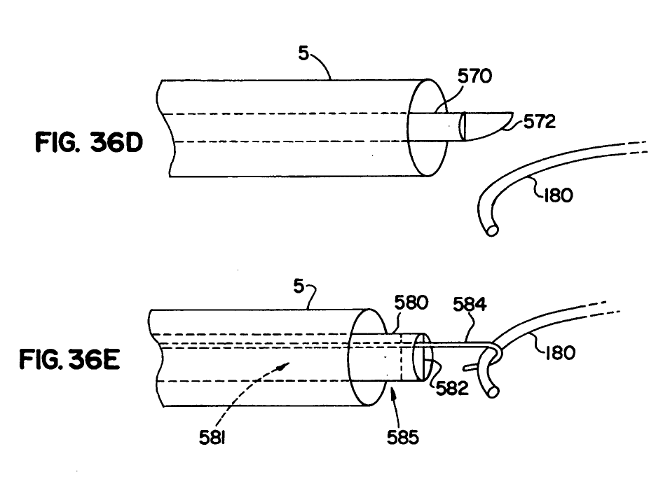

FIGS. 36(a)-36(c) depict an exemplary embodiment of a suture cutting mechanism 500. Cutting mechanism can be incorporated into a catheter used to deliver lock 400. Accordingly, cutting mechanism can be incorporated into inner catheter and/or outer catheter 420, as desired. Inner catheter 410 preferably includes a substantially stiff distal region 414a (including hard plastic and or a hypotube, for example) as described above. As depicted, inner catheter 410 holds apart two curved blades 510 that are mounted on pivots or hinges 512 in the lumen of outer catheter 420. The resting position of blades 510 is preferably a closed position, as depicted in FIG. 36(c). Blades 510 are preferably forced open by inner catheter 410 when inner catheter 410 is positioned between blades 410. As the inner catheter 410 continues to be withdrawn proximally as depicted in FIG. 36(b), blades 510 are no longer held apart by inner catheter 410 and are urged together fall on the suture 180. The cutting action can be completed by pushing inner catheter 410 distally such that blades 510 are received by inner lumen 416 of inner catheter 410 to force the blades 510 closed.

It will be appreciated that a variety of other means may be used to cut suture 180 to a desired length percutaneously. By way of example only, as depicted in FIG. 36(d) catheter 570 includes a blade 572 that can be used to sever a filament/suture 180. By way of further example, FIG. 36(e) depicts still a further suture cutting catheter 580 including an elongate body defining a lumen 581 therethrough having a retractable hook 584 adapted and configured to capture and drag suture 180 inside of lumen 581 to a blade 582 disposed near the distal end 585 of the catheter 580 to sever the suture 180.

In further accordance with the invention, still further alternative embodiments of devices and methods in accordance with the invention are provided.

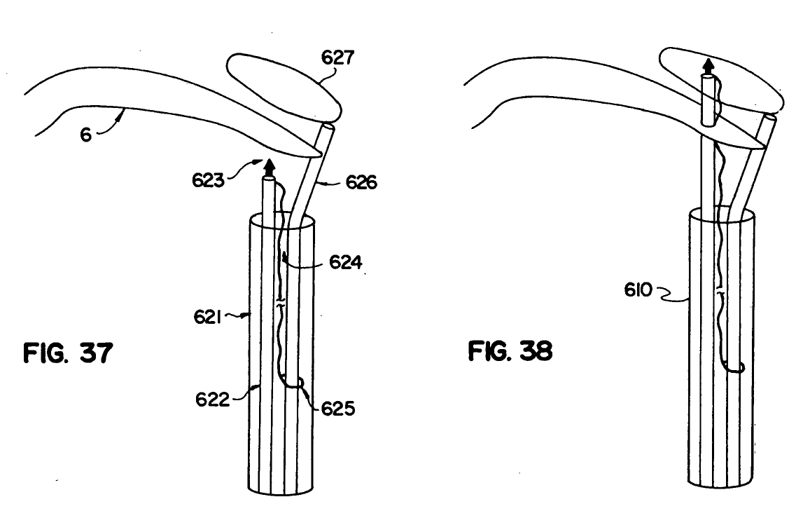

For purposes of further illustration and not limitation, as embodied herein and as depicted, in FIG. 37, a further embodiment of a device for ensnaring a prolapsing valve leaflet 6 is provided. A delivery or guide catheter 610 contains two smaller diameter catheters. One of these catheters 622 houses a needle tip 623 at its distal end, with attached suture 624. As in the previous embodiment, the suture is of sufficient length to traverse the length of the delivery catheter 621, and has a loop 625 located on the end. The second catheter 626 is a snare catheter, with the snare element 627 lying perpendicular to the catheter 626 when in the open position. This catheter 626 passes through the loop 625.

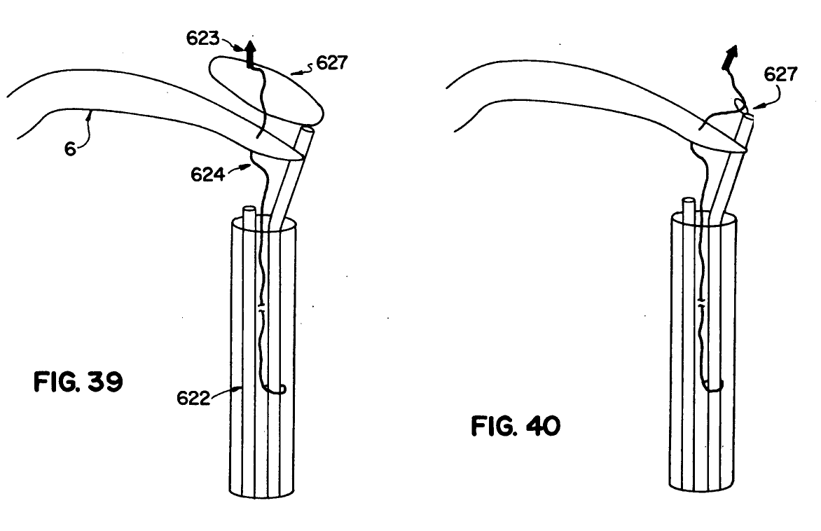

The snare catheter 626 is positioned beyond the valve leaflet (e.g., 6), then catheter element 622 is advanced through the leaflet and through the middle of snare element 627, as illustrated in FIG. 38. FIG. 39 illustrates the catheter 622 separated from the needle tip 623 and withdrawn back through the leaflet and into the delivery catheter 621, leaving the suture 624 through the leaflet and snare element. The snare element 627 is tightened, as illustrated in FIG. 40, to capture the suture 624.

The snare catheter 626 is then withdrawn into the delivery catheter 610, pulling the suture 624 through its own end loop 625, as shown in FIG. 41. The catheter 626 is withdrawn until the suture end with attached needle tip 623 are exteriorized from the patient and the loop assembly 625 forms a point of fixation on the leaflet 6. The delivery catheter 610 and its contents are then removed, leaving the suture 624 affixed to the leaflet 6 and the other end of suture 624 exteriorized from the patient for further manipulation as shown in FIG. 42.

An additional embodiment of a system utilizing such a snare method may be constructed as depicted in FIGS. 37-42 but without the use of suture loop 625. In brief, the suture used in such an embodiment is necessarily greater than twice the length of the delivery catheter 610 and after the end 623 of the suture is captured by the snare and withdrawn, the operator is left with two ends of suture. Both these ends will be passed through the papillary muscle anchor (e.g., 50) such that the resultant artificial chord 180 consists of two lengths of suture which passes through the leaflet at a single point.

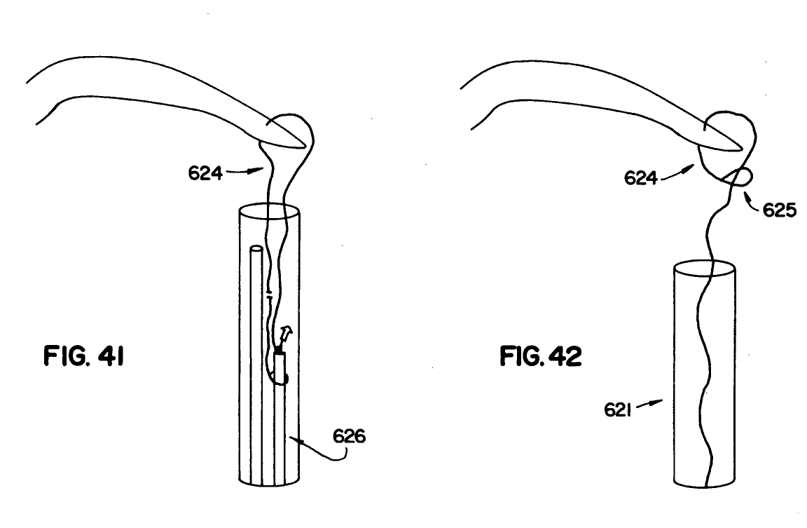

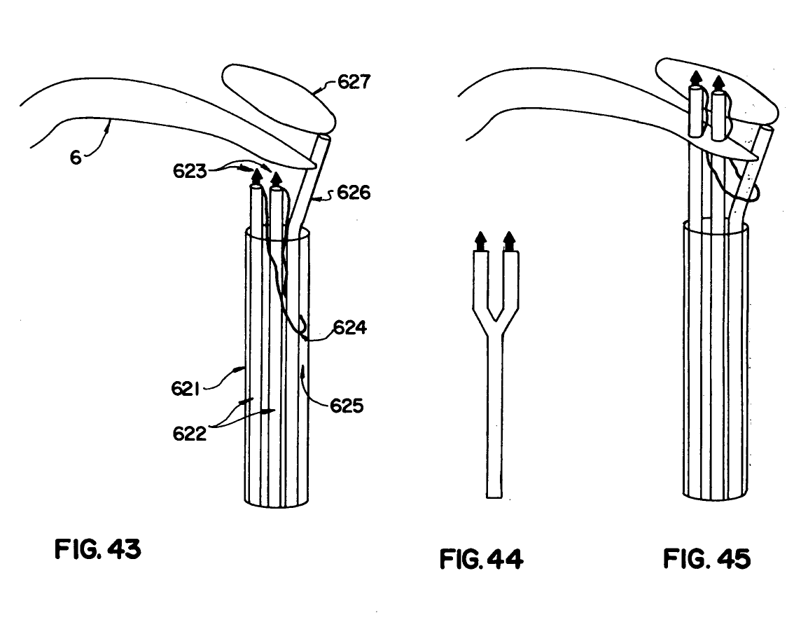

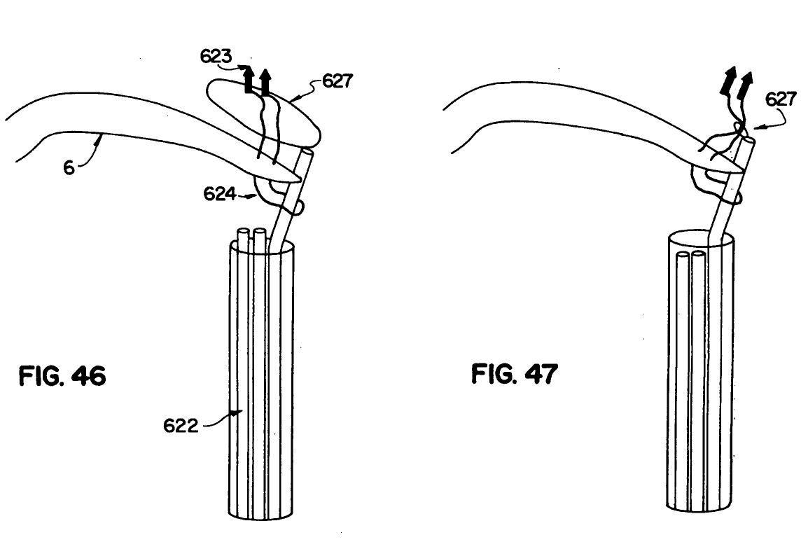

Specifically, and as depicted in FIGS. 43-49 yet another embodiment of the snare system and method is shown, whereby the delivery catheter 621 houses two internal catheter elements 622 that are each constructed with a needle tip 623 at an end thereof, with each tip 623 fastened to the end of a single length of suture material 624. The suture 624 loops around a third catheter element 626 that houses a snare 627. These catheter elements 622 may also be formed into a single bifurcated unit as depicted in FIG. 44. Each tip 623 is spaced at a defined distance from the other, most preferably from about 2 mm apart to about 5 mm apart. The delivery catheter 621 is adapted and configured to orient the tips 623 parallel to the leaflet edge so that each tip passes through the leaflet 6 at about the same distance from the edge. Once both tips 623 are through the leaflet and snared (FIGS. 46-49), withdrawing the snare catheter will cause the suture to pass through its own loop formed by passing around catheter 626. This loop will then pull snug against the leaflet edge, securely fastening the suture to the leaflet 6. The new chordae will then include two ends of suture brought through the papillary muscle anchor (e.g., 50).

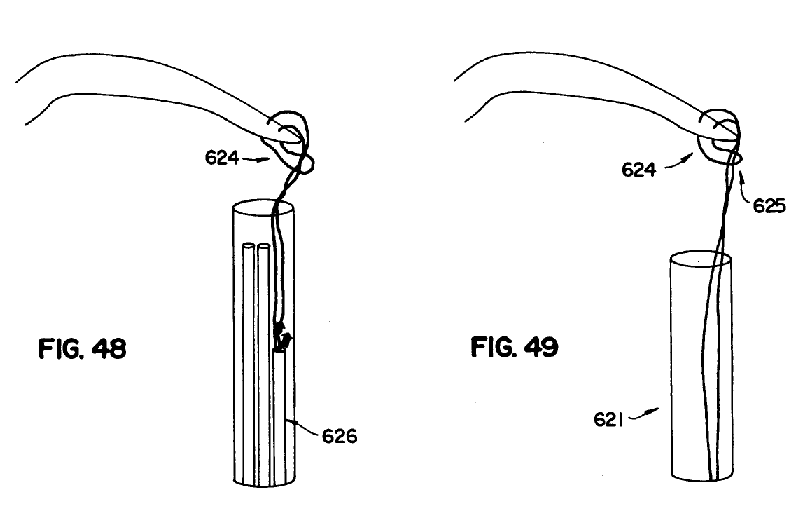

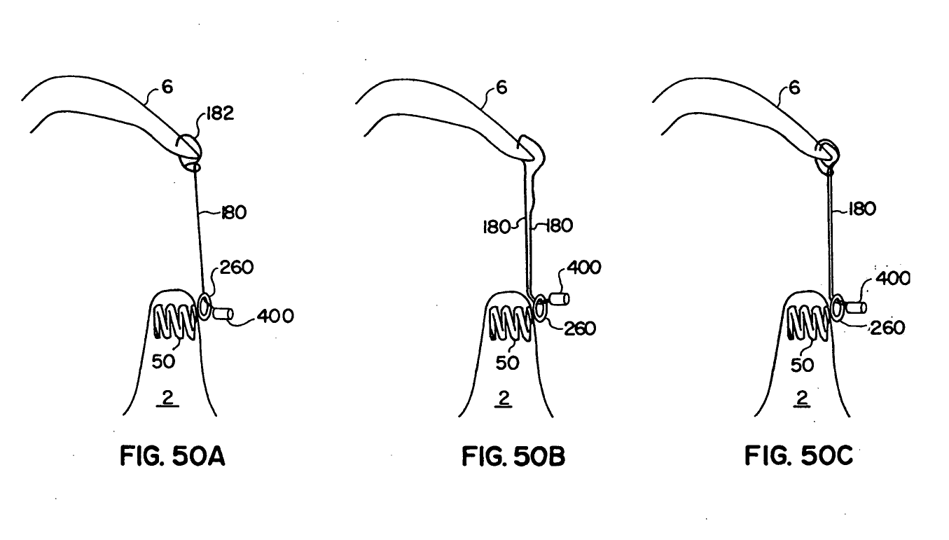

Thus, as embodied herein, the snare catheter embodiments describe several ways of implanting artificial chordae. This is illustrated in detail in FIGS. 50(a)-50(c). In FIG. 50(a), a single length of suture 180 passes through its own distal loop 182 before being secured to the papillary muscle anchor 50. In FIG. 50(b), a simple suture 180 directed through the leaflet 6 leaves two ends of suture 180 to secure to the papillary muscle anchor 50. In FIG. 50(c), two ends of the same suture 180 are passed through the leaflet 6, then pass through their own loop before being secured at the papillary muscle anchor 50.

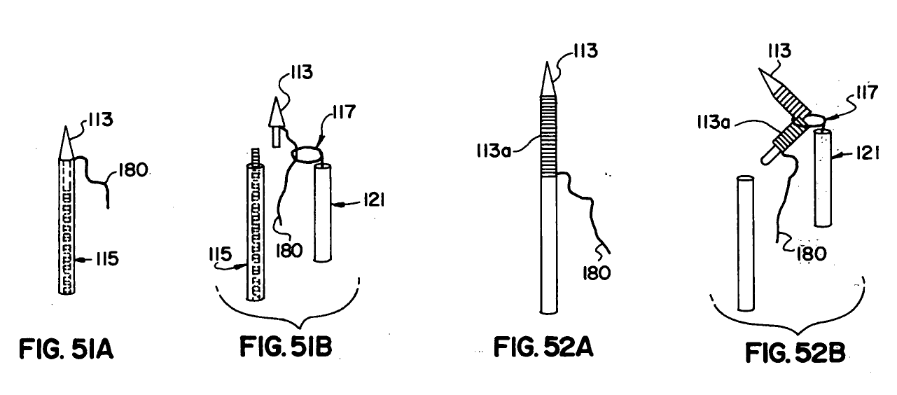

As depicted in FIGS. 51-52, by way of still further example, designs for the leaflet delivery catheter 110 using the snare approach described herein can include a simple point assembly 113 with attached suture 180 where the suture is captured by the snare or a tip assembly that itself is grasped by the snare. In the former, an example of which is illustrated in FIG. 51, a catheter 110 a houses a sharp tip 113 with attached suture 180. The lumen of catheter 110 houses a pushrod or stiff wire 115. Once passed through the leaflet 6 and open snare 117, the pushrod 115 ejects the tip 113 from catheter 110, allowing suture 180 to be captured by the snare without entraining catheter 110. The tip assembly depicted in FIG. 52 includes a sharp tip element 113 with attached segment 113(a) that can detach from catheter 110. The segment 113(a) can be, for example, a tightly wound coil or a segment of material with non-brittle properties such that it remains straight when pushed from the end, but will fold when grasped in its mid-section. For example, a metal coil would be suitable. The suture 180 can be attached either to the tip 113 or segment 113(a). Thus, once passed through the leaflet and grasped by the snare 117, the assembly 113(a) can easily be folded in half (since it is made from a coil) and be pulled into the lumen of snare catheter 121 before being withdrawn through the body, thus increasing the safety of pulling the sharp tip 113 back through a guide catheter (e.g., 5).

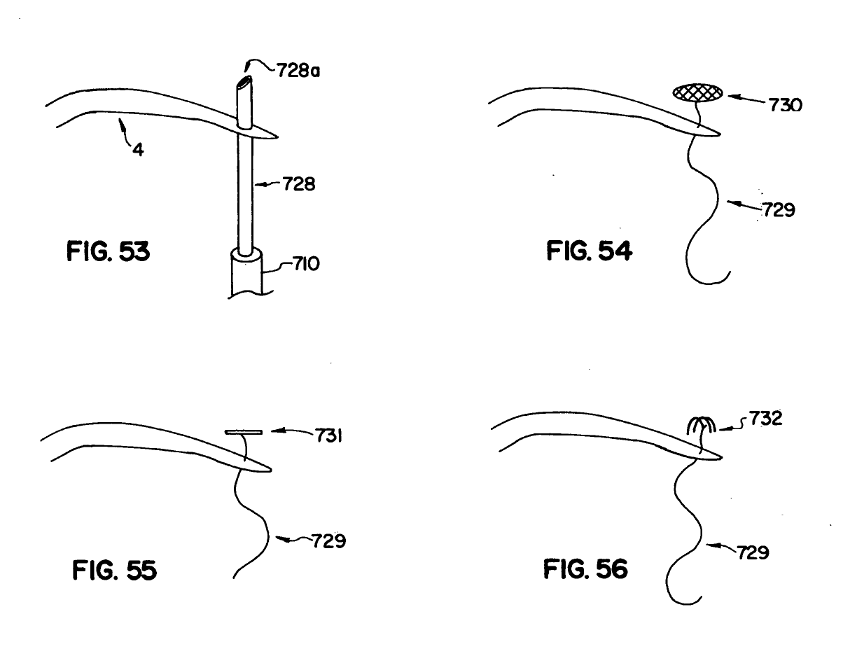

An additional embodiment of a leaflet capture device include the use of a catheter 710 with hollow-bore needle 728 at its distal end that traverses the valve leaflet (e.g., 4), as illustrated in FIG. 53. Contained within this catheter and needle assembly are the suture 729 and an anchoring element 730, as depicted in FIG. 54. The anchoring element 730 may be a compressed pledget of felt, fabric, or similar material with either nonabsorbable or bioabsorbable properties. This will fit within the delivery catheter 710 and needle assembly 728, be deployed by a pushrod element, then either unfurl or expand to prevent it from pulling back through the tract created by the needle. The needle 728 is withdrawn once the pledget is deployed, leaving the pledget anchor on the far side of the leaflet and the suture 729 exiting the near side of the leaflet. This anchor can also include a flat disc of metal or similarly rigid material that in its resting state assumes a flat shape with the suture affixed at its mid point. The disc can be rolled around the suture such that its profile will fit within the needle 728 until it is deployed as shown in FIG. 54.

Moreover, multiple possible anchors can be deployed using this method, such as a T-bar anchor 731 that has the suture fixed at its mid-point and is fashioned of a rigid material. Such an anchor 731 preferably will lie vertical within the needle shaft, then rotate horizontal when deployed as depicted in FIG. 55. As depicted in FIG. 56, a suitable anchor 732 may also be fashioned of a material with shape memory properties, such as nitinol. Such an anchor can lie compressed within a lumen 728a of the needle assembly 728 until deployed from the needle tip, then assume its non-compressed state to act as an anchor 732.

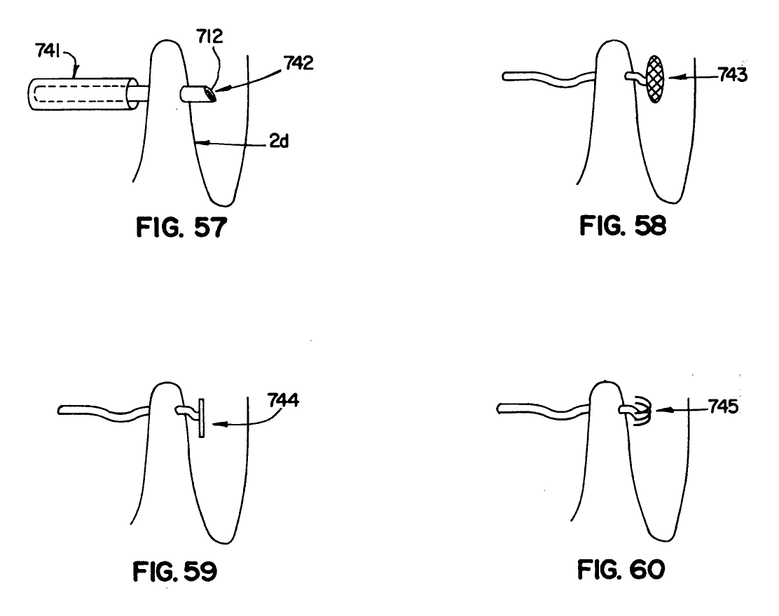

As depicted in FIGS. 57-60 in yet another embodiment, the use of a hollow-bore needle 742 affixed to a catheter 710 for placement of papillary muscle anchor is illustrated. Needle 742 is passed through the papillary muscle 2 after which an anchor (e.g., 743, 744, 745) is pushed out of lumen 712 of the catheter 710 and needle 742 to deploy on the distal side 2d of the papillary muscle 2. By way of example, anchors may be constructed of pledget material or a flat disc 743 as depicted in FIG. 58. A T-bar 744 may also be used as depicted in FIG. 59, or an anchor 745 of shape memory material such as nitinol may be used as depicted in FIG. 60.

A further embodiment of papillary muscle anchoring method may be by direct suture of the papillary muscle 2 using a device similar to that in FIG. 5 or FIG. 37.

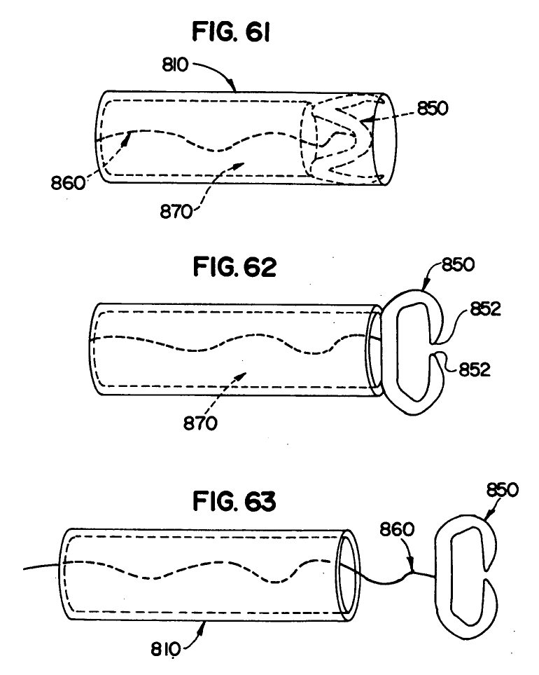

In accordance with another embodiment, as depicted in FIGS. 61-63, an anchor 850 is provided that may be used for either leaflet capture or as a papillary muscle anchor. Anchor 850 is formed of a shape memory material such as nitinol or a material with analogous physical properties. Anchor 850 can be constrained in a catheter 810, with attached suture 860 and a solid or hollow-bore push rod 870. Once the delivery catheter 810 is positioned firmly against the leaflet 4, 6 or papillary head 2, the pushrod 870 is extended, driving the sharp points 852 of the staple anchor 850 into the target. Once free of the constraints of the outer delivery catheter, the anchor 850 assumes its resting memory position as depicted in FIG. 62, capturing the leaflet or papillary muscle. The delivery catheter 810 is then withdrawn as depicted in FIG. 63, leaving the anchor 850 and attached suture 860.

While the systems depicted herein are generally described for repair of a regurgitant and prolapsing mitral or tricuspid valve, these systems may also be used for valve repair in other circumstances.

For example, restricted leaflet motion, as may occur in dilated, ischemic, or rheumatic disease, may be addressed by one of several means. A method treating this condition will now be described. First, an artificial chordae may be placed as described herein, and the native restrictive chordae may then be cut, releasing the tethered portion of the valve leaflet. This will result in a valve leaflet with an increased range of motion.

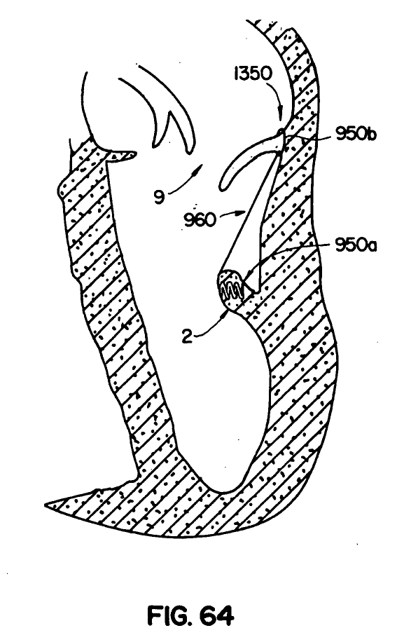

With reference to FIG. 64, by way of further example, an anchor 950a can be placed to the papillary muscle 2 and a second anchor 950b can be applied to the mitral valve annulus 1350, then the suture 960 joining them may be tightened to alter the geometric relationship of papillary muscle 2 to mitral valve 9, loosening the tethered native chordae.

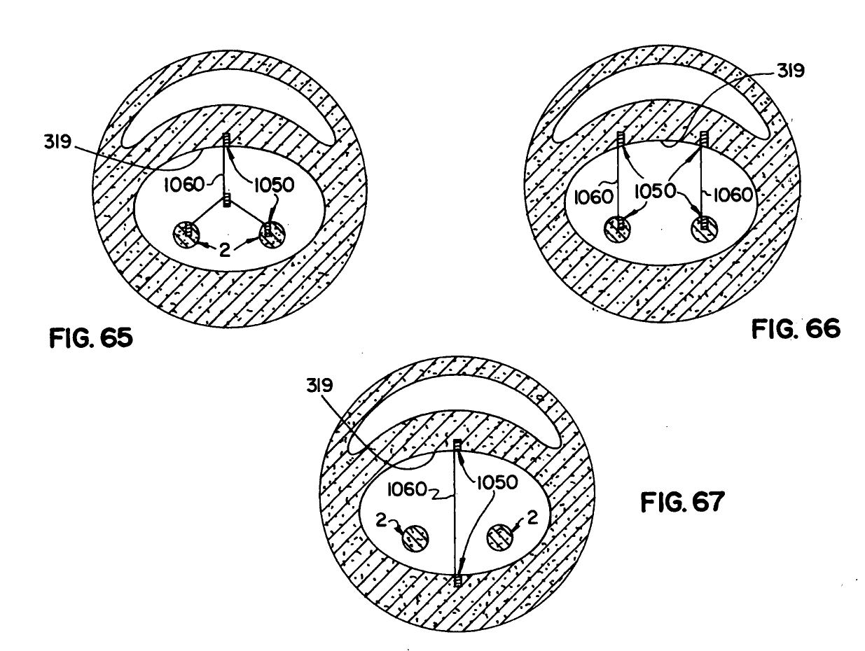

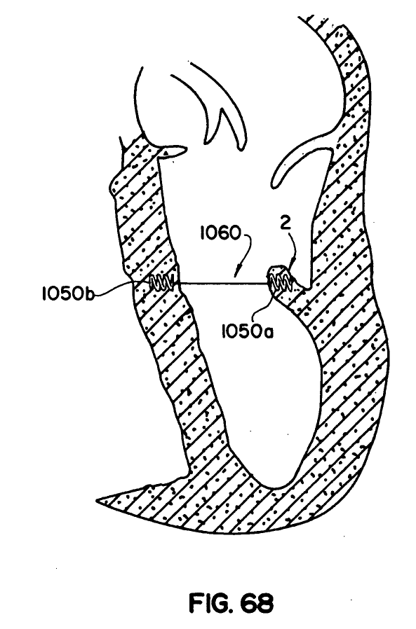

As depicted in FIGS. 65-68, anchors 1050 may be implanted into one or both papillary muscles 2 and an additional anchor or anchors 1050 may be implanted in the ventricular septum 319, then the suture(s) 1060 between these anchor points may be tensioned and fastened in a manner analogous to that described for the artificial chordal adjustment. This technique is illustrated in a long-axis left ventricular view in FIG. 68 and in short-axis left ventricular views in FIGS. 65-67. This will create a catheter-delivered endoventricular restraint, reducing the septal-lateral dimension of the ventricle and reversing valvular regurgitation due to ventricular dilation.

The devices and systems described herein can be used for percutaneous repair of mitral valves arising from degenerative mitral valve disease as well as other causes, such as enlarging of the heart. Systems made in accordance with the teachings herein can have significant utility among both interventional cardiologists, who have traditionally applied catheter-based techniques, and surgeons, who have traditionally treated severe mitral valve disease. Moreover, the systems devices and methods described herein update the traditional replacement of mitral valve chordae using open-heart surgery and will complement the current valve repair approaches currently in development and testing.

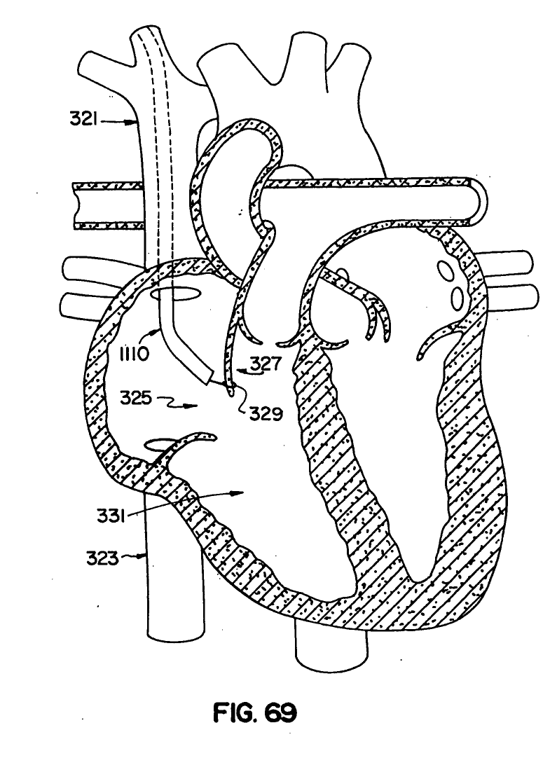

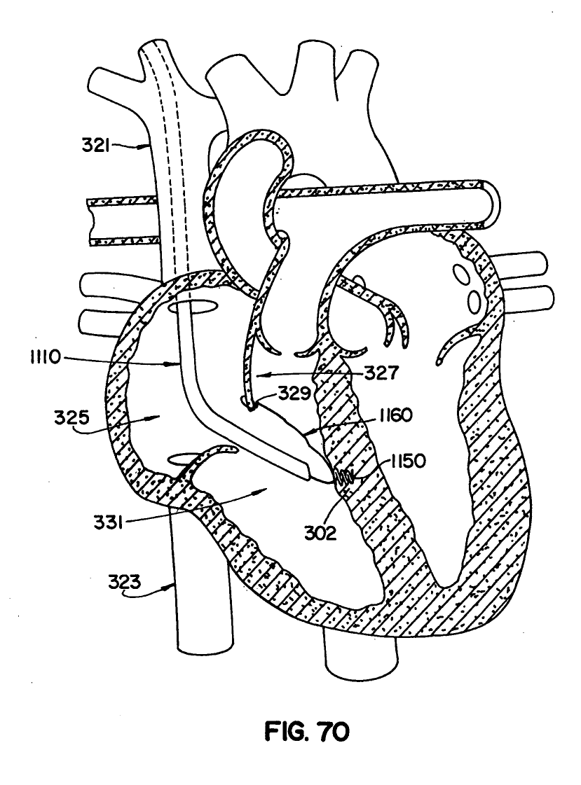

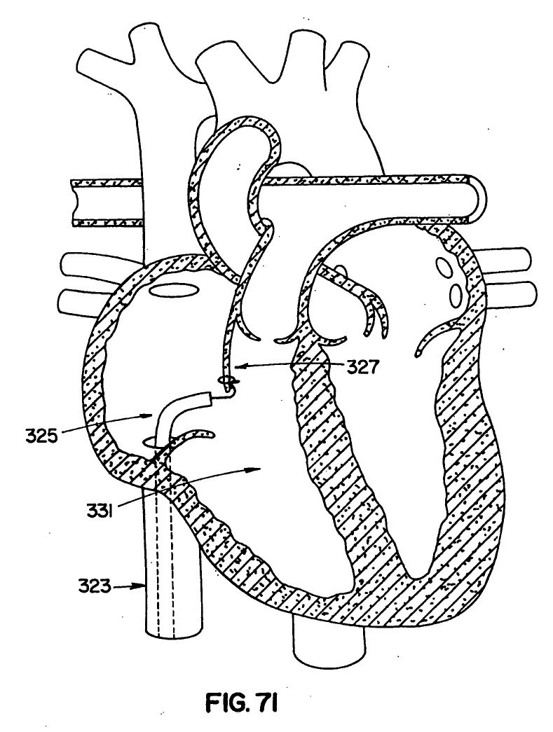

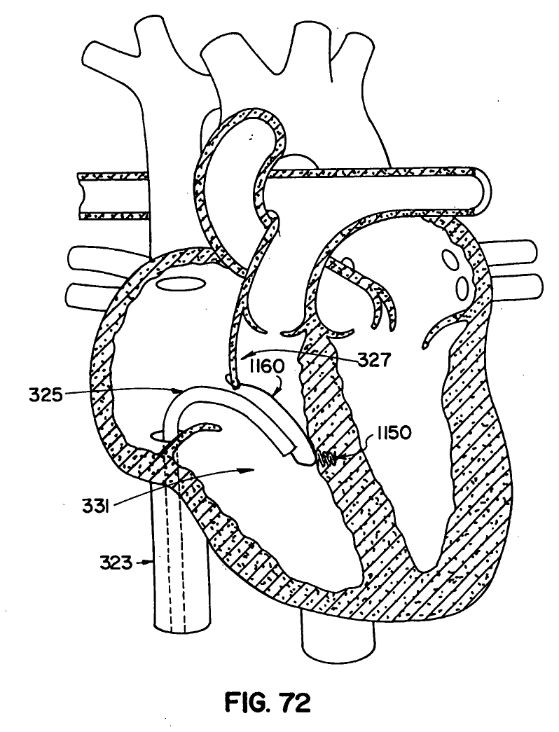

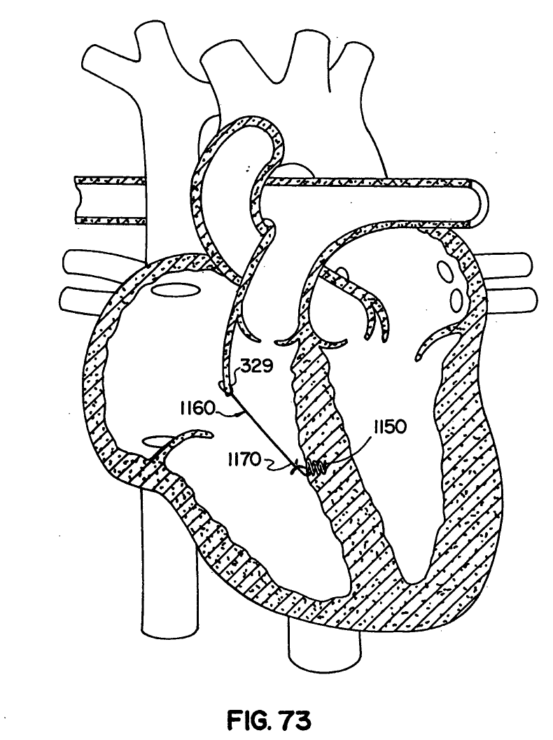

Systems and methods in accordance with the present invention may also be used to repair the tricuspid valve by positioning in the right heart. This may be achieved by passing a catheter through the venous system to the right atrium, then directing the system toward the tricuspid valve and subvalvular apparatus. This technique is illustrated in FIGS. 69-73. The catheter 1110 is directed through either the superior vena cava 321 as depicted in FIGS. 69-70 or the inferior vena cava 323 as depicted in FIGS. 71-72 to the right atrium 325 and directed at a leaflet 329 of the tricuspid valve 327. The suture 1160 is affixed to the leaflet edge as previously described for the mitral valve 9, then an anchor 1150 is placed in the papillary muscle 2 by advancing the catheter 1110 into the right ventricle 331. The length of suture from the tricuspid valve 327 to the papillary muscle 2 is adjusted by the operator under image guidance until the desired length of neochorda(e) is created to reduce or eliminate the tricuspid regurgitation. The suture 1160 is fixed at this length with a fastener 1170 and the suture tail cut, leaving the fixed length of neochorda(e) as depicted in FIG. 73.

By way of still further example, a method and associated system are provided for facilitating coaptation between leaflets of a cardiac valve of a patient by at least partially folding over a valve leaflet, and securing the valve leaflet in place.

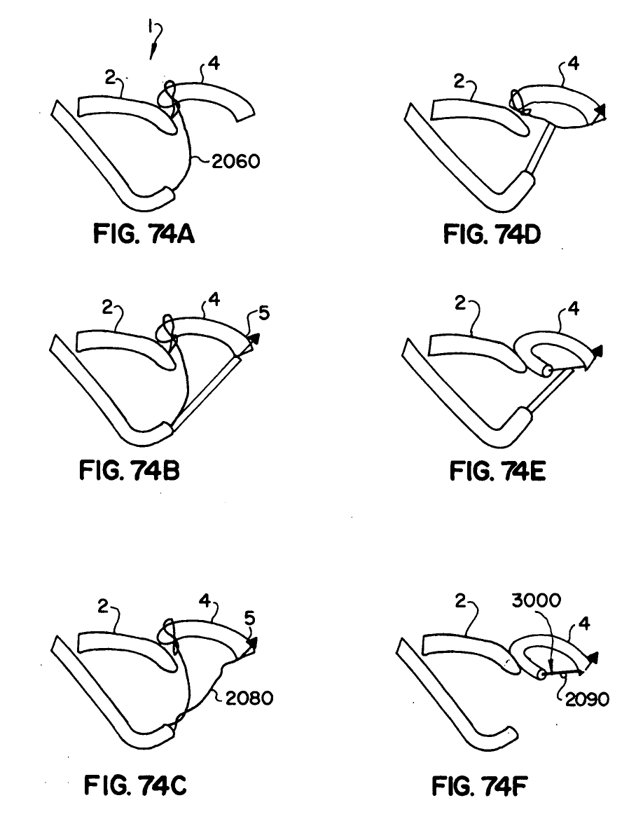

For purposes of illustration and not limitation, as embodied herein and as depicted in FIGS. 74(a)-74(f), a method and associated system are depicted herein for achieving valve leaflet coaptation. As depicted in FIG. 74, leaflets 2, 4 of valve 1 are not properly coapting. Specifically, the leaflets are too large in order to close valve 1 properly. Accordingly, to “shorten” on the of the leaflets 4, a suture 2060 is passed through leaflet 4 near an edge thereof to capture leaflet 4, as depicted in FIG. 74(a). Next, an anchor is deployed connected to a second end of suture material 2080, and anchored into a wall of vascular tissue 5. The sutures 2060, 2080 are drawn taut, and a clip 2090 is affixed to create a neochordae 3000. The “resized” leaflet 4 now aligns well with, and coapts with, leaflet 2, permitting valve 1 to close properly, thereby decreasing the risk of regurgitation.

As will be appreciated, the method as embodied in FIG. 74 can be practiced on any suitable cardiac valve (e.g., mitral, tricuspid and the like) and can be performed by connecting two sutures 2060, 2080, as well as by using a single suture and anchor mechanism as described herein above.

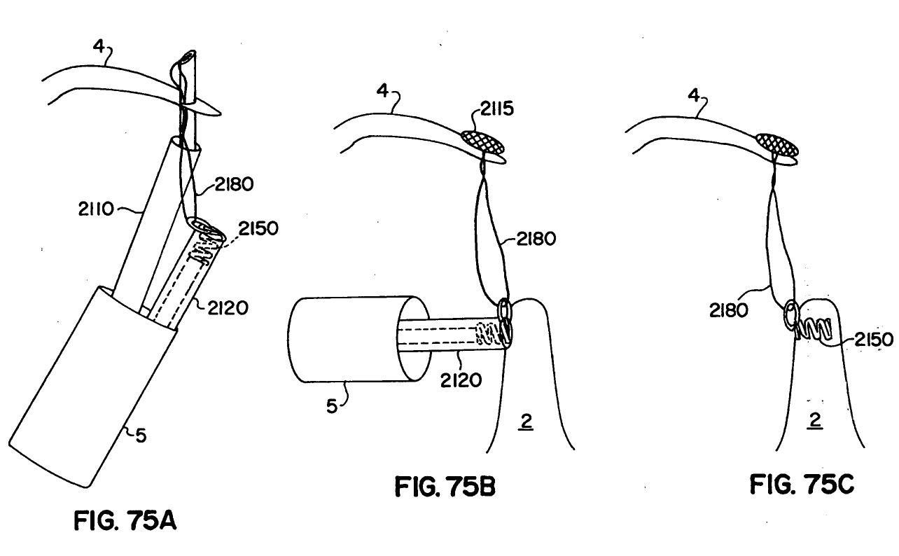

By way of further example, it is possible to use a modification of the components listed in this application to implant premeasured, fixed-length chordae rather than adjustable chordae. The potential advantage of this technique is elimination of need for a fastening and cutting system. For purposes of illustration and not limitation, an exemplary embodiment of such a system and associated are depicted in FIGS. 75(a)-75(c).

In accordance with this embodiment of the invention, measurement of appropriate chordal length can be done pre- or intra-procedure using an imaging modality such as echocardiography. The appropriate length of the chordae 2180 may then be determined accordingly, such as from the anterior leaflet 4 to the papillary 2. However, as will be appreciated, such a method can be carried out for myriad applications within the patient's lumenal system. Accordingly, one or more catheters 2110 as appropriate having chordae/filaments/sutures 2180 with varying lengths preloaded on the catheter 2110, or the operator could prefix a chordal length before inserting the device 2110 into a patient. If desired, guide catheter 5 may contain a plurality of delivery catheters—one catheter 2110 with a leaflet anchor 2115 and one 2120 with a papillary muscle anchor 2150, both anchors joined by the length of chord 2180, which could be, for example, a single length of suture material or a loop, as depicted in FIG. 75. The leaflet anchor 2115 may be deployed and then the papillary or ventricular anchor 2150 may be deployed, leaving the chord 2180 anchored at both locations. The operator may be provided with flexibility in the effective chordal length during implantation in choosing the exact location for papillary or ventricular anchoring. Fine-tuning of the chordal length can be performed under image-guidance (e.g. echocardiography) akin to the adjustable chord method.

It will be further appreciated that the present invention embraces percutaneous placement of neochordae anywhere in the vascular system, anchoring anchors into any suitable tissue. For example, while significant illustrations were described herein affixing anchors (e.g., 50) to papillary muscle tissue 2, it will be appreciated that anchors can be affixed to any suitable tissue, including, e.g., the ventricular wall of the heart.

It will also be understood that while multiple catheters have been described herein (e.g., for leaflet capture, for anchor installation, for retainer application, for lock application, for severing filamentary material), any two or more of these and other suitable functions may be combined in a single catheter.

The methods and systems of the present invention, as described above and shown in the drawings, provide for a system and method of cardiac valve repair with superior advantages over prior art approaches. These advantages include, by way of example only, adjusting the size of artificial chordae while the patient's heart is beating. This is advantageous because it greatly increases the chances for successful procedures, and eliminates the need for open heart surgery, permitting cardiac valve repair on an out patient basis.

It will be apparent to those skilled in the art that various modifications and variations can be made in the system and method of the present invention without departing from the spirit or scope of the invention. Thus, it is intended that the present invention include modifications and variations that are within the scope of the appended claims and their equivalents.

What is claimed is:

1. A method of repairing a cardiac valve, comprising:
a) introducing a catheter through a patient's vasculature into the patient's heart;
b) advancing a distal end of the catheter proximate a leaflet of a cardiac valve of the patient;
c) using the catheter to direct a first portion of a filament through the leaflet to capture the leaflet; and
d) applying tension to the filament to adjust the function of the cardiac valve.

2. The method of claim 1, further comprising adjusting a length of the filament to vary the function of the cardiac valve.

3. The method of claim 2, wherein the length of the filament is adjusted while the patient's heart is beating.

4. The method of claim 3, further comprising viewing the patient's heart using an imaging technique while the length of the filament is adjusted to determine a desired length for the filament.

5. The method of claim 4, further comprising directing a second portion of the filament toward a second location within the patient's vasculature.

6. The method of claim 5, wherein the second portion is directed to the second location by:
a) implanting an anchor in cardiac tissue in the second location; and
b) directing the second portion of the filament through a portion of the anchor.

7. The method of claim 6, wherein the length of the filament is adjusted by applying tension to the filament through the anchor.

8. The method of claim 7, wherein tension is applied to the filament by disposing a distal end of the catheter against the anchor and pulling on the filament using the anchor as a fulcrum.

9. The method of claim 7, further comprising applying a lock to the filament to prevent the second portion of the filament from disengaging from the anchor.

10. The method of claim 9, wherein the lock is applied to a portion of the filament that is on an opposite side of the anchor from the first portion of the filament.

11. The method of claim 9, further comprising severing the filament on a side of the lock opposite the anchor.

12. The method of claim 1, wherein the filament includes suture material.

13. The method of claim 12, wherein the suture material includes a monofilament.

14. The method of claim 12, wherein the suture material includes a polyfilament braided material.

15. The method of claim 12, wherein the suture material includes material selected from the group consisting of polypropylene, polyester, nylon, and silk.

16. The method of claim 12, wherein the suture material includes radiopaque material.

17. The method of claim 12, wherein the suture material has echodense properties to facilitate visualization thereof using fluoroscopic or echocardiographic imaging techniques.

18. The method of claim 17, wherein the suture material includes radiopaque material and echodense material.

19. The method of claim 12, wherein the suture material includes expanded PolyTetraFluoroEthylene.

20. The method of claim 19, wherein the suture material includes nodes and fibrils adapted to facilitate tissue ingrowth therein.

21. The method of claim 6, wherein the anchor includes at least one barb, the barb being adapted and configured to resist backout of the anchor from tissue in which the anchor is implanted.

22. The method of claim 21, further comprising deploying the at least one barb from an undeployed to a deployed position after implanting the anchor.

23. The method of claim 1, wherein the catheter is introduced into the patient's vasculature through a guide catheter.

24. The method of claim 1, wherein the catheter is introduced into the patient's vasculature over a guidewire.

25. The method of claim 23, wherein the catheter is introduced into the patient's vasculature over a guidewire.

26. The method of claim 1, wherein the cardiac valve is the patient's mitral valve.

27. The method of claim 5, wherein the second location is a papillary muscle portion of the patient.

28. The method of claim 1, wherein the cardiac valve is the patient's tricuspid valve.

29. The method of claim 1, further comprising:
a) using the catheter to direct a first portion of a second filament through the leaflet to capture the leaflet; and
b) applying tension to the second filament to further adjust the function of the cardiac valve.

30. The method of claim 4, wherein the imaging technique is selected from the group consisting of echocardiography and fluoroscopy.

31. The method of claim 1, further comprising affixing a second portion of the filament to a first portion of a second filament.

32. The method of claim 31, further comprising attaching a second portion of the second filament to an anchoring location within the patient's heart.

33. The method of claim 32, wherein the second filament is attached to the anchoring location by affixing it to an anchor embedded in cardiac tissue.

34. The method of claim 33, wherein the anchor is embedded in a papillary muscle.

35. The method of claim 6, wherein the anchor is implanted by the same catheter used to deliver the filament.

36. The method of claim 9, wherein the lock is applied to the filament using the same catheter used to deliver the filament to the valve leaflet.

37. The method of claim 31, wherein the filaments are affixed to each other using the same catheter used to deliver the filament to the valve leaflet.

38. The method of claim 11, wherein the filament is severed using the same catheter used to deliver the filament to the leaflet.

39. A method of adjusting the geometry of a patient's heart, comprising:
a) introducing a catheter through a patient's vasculature into the patient's heart;
b) advancing a distal end of the catheter proximate a first portion of the interior of the patient's heart;
c) using the catheter to attach a first portion of a filament to the first portion;
d) using the catheter to attach a second portion of the filament to a second portion of the interior of the patient's heart; and
e) applying tension to the filament to adjust the geometry of the patient's heart.

40. The method of claim 39, further comprising setting the final length of the filament.

41. The method of claim 40, wherein the final length of the filament is established by applying a retainer to the filament.

42. The method of claim 39, wherein the geometry of the patient's heart is adjusted to decrease mitral valve regurgitation.

43. The method of claim 42, wherein mitral valve regurgitation is decreased by reducing the septal-lateral dimension of the patient's ventricle.

44. The method of claim 39, wherein the geometry of the patient's heart is adjusted to improve operation of the patient's tricuspid valve.

45. A catheter adapted and configured to deliver a filament through a patient's vasculature into the patient's heart proximate a leaflet of a cardiac valve of the patient, the catheter comprising:
a) an elongate body having a proximal end and a distal end;
b) a filament in operable association with the elongate body; and
c) a deployable penetrator in operable association with the elongate body, the deployable penetrator being adapted and configured to be deployed through a portion of a patient's valve leaflet to facilitate capture of the leaflet by the filament.

46. The catheter of claim 45, further comprising a leaflet grasping portion adapted and configured to hold the patient's valve leaflet in place to facilitate deploying the penetrator through the leaflet.

47. The catheter of claim 46, wherein the leaflet grasping portion hold's the patient's valve leaflet in place at least in part by applying suction to the valve leaflet.

48. The catheter of claim 46, wherein the leaflet grasping portion is pivotally mounted proximate a distal region of the catheter.

49. The catheter of claim 46, wherein the leaflet grasping portion includes two pivotally mounted arms mounted on a distal region of the catheter.

50. The catheter of claim 45, wherein the filament is a first filament, and the catheter further includes a second filament adapted and configured to receive the penetrator.

51. The catheter of claim 50, wherein the second filament includes a cuff adapted and configured to receive the penetrator.

52. The catheter of claim 50, wherein the second filament further includes a loop therein for receiving the first filament therethrough.

53. The catheter of claim 52, wherein the catheter is adapted and configured to permit the second filament to be pulled through the leaflet to permit the loop to form a knot about the leaflet to capture the leaflet.

54. The catheter of claim 53, wherein the catheter is further adapted and configured to permit a free end of the second filament to be exteriorized from the patient.

55. The catheter of claim 54, wherein the second filament is exteriorized through a lumen of the catheter.

56. The catheter of claim 45, wherein the catheter further includes an anchor deployment portion for deploying an anchor into cardiac tissue of the patient.

57. The catheter of claim 45, wherein the catheter further includes a retainer applicator for applying a retainer to the filament.

58. The catheter of claim 57, wherein the retainer applicator is adapted to apply a retainer to the filament proximate an anchor embedded in the patient's vasculature.

59. The catheter of claim 57, wherein the retainer applicator is adapted to apply a retainer to a plurality of filaments to secure the plurality of filaments to each other.

60. The catheter of claim 45, wherein the catheter further includes a blade disposed thereon adapted and configured to severe the filament.

61. A catheter for applying an anchor into cardiac tissue of a patient, comprising:
a) an elongate outer body having a proximal end, a distal end and defining a lumen therethrough;
b) an torquable elongate inner body movably disposed within the lumen of the outer body, the inner body having a fitting for receiving an anchor therein.

62. The catheter of claim 61, wherein the inner body is formed at least in part from a hypotube.

63. The catheter of claim 62, wherein the inner body has a varying flexibility along its length.

64. The catheter of claim 63, wherein the varying flexibility is provided at least in part by a plurality of cuts formed in the inner body.

65. The catheter of claim 63, wherein the outer body has varying flexibility along its length.

66. The catheter of claim 65, wherein the varying flexibility is provided at least in part by at least one stiffening wire formed into the outer body.

67. The catheter of claim 61, further comprising a guide lumen formed on the outer body having a filament inlet port proximate the distal end of the outer body and a filament exit port proximal of the filament inlet port for receiving a filament therethrough.

68. The catheter of claim 67, wherein the filament exit port is substantially close to the distal end of the outer body.

69. The catheter of claim 61, further comprising;
a) an anchor disposed in the fitting; and
b) a filament disposed about the inner body, the filament being operatively associated with the anchor.

70. The catheter of claim 61, further including a torquable handle attached to the proximal end of the inner body for applying a torque to the inner body.

71. The catheter of claim 61, further comprising a steering mechanism adapted and configured to steer a distal end of the catheter.

72. The catheter of claim 61, further comprising an anchor guide disposed on an inside surface of the outer body, the anchor guide being adapted and configured to guide the anchor during installation of the anchor.

73. The catheter of claim 72, further comprising a helical anchor disposed in the fitting, and wherein the anchor guide facilitates rotation of the anchor while it is being installed.

74. The catheter of claim 73, wherein the anchor guide urges the anchor distally as it is rotated with respect to the outer body by the inner body.

75. The catheter of claim 61, further comprising an anchor disposed in the fitting.

76. The catheter of claim 75, wherein the anchor includes at least one barb disposed thereon to resist backout of the anchor after it has been implanted.

77. The catheter of claim 75, wherein the anchor is formed from shape memory material.

78. A catheter for fastening together a plurality of filaments, comprising:
a) an elongate body having a proximal end and a distal end;
b) a guide passage for receiving a plurality of filaments to be fastened together;
c) a fastener applicator disposed proximate the distal end of the elongate body for applying a fastener to the plurality of filaments received by the guide passage; and
d) an actuator disposed proximate the proximal end of the elongate body operably coupled to the fastener applicator to facilitate fastening the plurality of filaments.

79. The catheter of claim 78, wherein the guide passage includes a distal opening proximate the distal end of the elongate body for receiving the filaments, and a proximal exit opening spaced proximally from the distal end of the elongate body that permits passage of the filaments therethrough.

80. The catheter of claim 79, wherein the proximal exit opening is substantially closer to the distal end of the elongate body than the proximal end of the elongate body.

81. A catheter for positioning a lock on a filament comprising:
a) an inner member having a proximal end, a distal end and defining a lumen at least partially therethrough, the lumen being adapted and configured to receive a filament therethrough;
b) a filament lock biased to change from a first, relatively open state to a second, relatively closed state, the filament lock being disposed on a substantially rigid portion of the inner member, the inner member being sufficiently rigid to prevent the filament lock from changing from the first state to the second state; and
c) an outer member having a proximal end, a distal end and defining a lumen at least partially therethrough; the lumen of the outer member being adapted and configured to movably receive the inner member, the lumen being sufficiently small to prevent the filament lock from entering the lumen when positioned on the inner member.

82. The catheter of claim 81, wherein the filament lock includes a helical body wound about the inner member biased to contract in radius from the first state to the second state.

83. The catheter of claim 81, wherein the filament lock includes a pair of jaws hingedly connected that are biased to close on the filament.

84. The catheter of claim 81, wherein the filament lock includes a plurality of legs that are biased to close on the filament.

85. The catheter of claim 81, wherein the filament lock includes a substantially tubular body made from shape memory material that is adapted and configured to contract about the filament when exposed to the temperature of the body of the patient.

86. The catheter of claim 81, further comprising a pair of jaws adapted and configured to crimp the filament lock on the filament.

87. The catheter of claim 81, further comprising a filament exit port disposed in each of the inner member and outer member to permit passage of the filament therethrough.

88. The catheter of claim 87, wherein the filament exit ports are proximate the distal end of the inner and outer members.

89. An anchor adapted and configured to be anchored in cardiac tissue of a patient, comprising:
a) an anchoring portion having a proximal end and a distal end, the anchoring portion being adapted and configured to be anchored into cardiac tissue of a patient; and
b) a filament lock disposed at the proximal end of the anchoring portion, the filament lock being biased to change from a first, relatively open state to a second, relatively closed state when disposed about a filament, the filament lock defining a lumen therethrough for receiving an locking onto a filament.

90. The anchor of claim 89, wherein the anchor is formed at least in part from a shape memory material.

90. The anchor of claim 90, wherein the shape memory material includes nitinol.

91. The anchor of claim 89, further including a coupling member affixed to the anchoring portion, the coupling member defining a lumen therethrough for receiving a filament.

92. A catheter for severing a filament inside of a patient's vasculature, comprising:
a) an inner member having a proximal end, a distal end, and defining a lumen therethrough, the lumen being adapted and configured to receive a filament therethrough;
b) an outer member having a proximal end, a distal end and defining a lumen therethrough, the lumen of the outer member being adapted and configured to receive the inner member; and
c) a pair of substantially arcuate cutting jaws pivotally mounted inside the lumen of the outer member in a wall of the outer member; the jaws being biased to close about and sever the filament; the jaws being held apart by the inner member when the inner member is positioned between the jaws, wherein the filament is severed when the inner member is moved out of alignment with the cutting jaws.

93. A method of treating a cardiac valve, comprising:
a) introducing a catheter through a patient's vasculature into the patient's heart;
b) advancing a distal end of the catheter proximate a leaflet of a cardiac valve of the patient;
c) using the catheter to direct a first portion of a filament through the leaflet to capture the leaflet;
d) attaching a second end of the filament to cardiac tissue proximate an annulus of the cardiac valve;
e) applying tension to the filament to cause the leaflet of the cardiac valve to fold over onto itself until coaptation of the leaflet is established with an adjoining valve leaflet.

94. The method of claim 93, wherein the leaflet is attached to the annulus of the cardiac valve using a plurality of connected filaments joined by a retainer.

95. A system for treating a cardiac valve of a patient, comprising:
a) a first catheter for directing a first portion of a filament through a leaflet of a patient's cardiac valve to capture the leaflet; and
b) a second catheter for implanting an anchor into cardiac tissue of the patient displaced from the leaflet.

96. The system of claim 95, further comprising a third catheter for applying a filament lock to the filament after tension has been applied to the filament to change the operation of the cardiac valve.

97. The system of claim 95, wherein the second catheter is adapted and configured to receive the filament from the first catheter and directs the filament through the anchor.

98. The system of claim 96, wherein the third catheter further includes a blade for severing the filament after the filament lock has been applied.

99. The system of claim 95, wherein the filament includes suture material.

100. The method of claim 99, wherein the suture material includes material selected from the group consisting of polypropylene, polyester, nylon, silk and expanded PolyTetraFluoroEthylene.

101. A method of implanting a filament in a lumenal system of a patient; comprising:
a) introducing a catheter through a lumenal system of a patient to a location in the patient to be treated;
b) advancing a distal region of the catheter proximate a first location;
c) attaching a first portion of a filament to the first location using the catheter;
d) advancing the distal region of the catheter proximate a second location; and
e) attaching a second portion of the filament to the second location using the catheter.

102. The method of claim 101, wherein the location to be treated is inside of the patient's heart.

103. The method of claim 101, wherein the filament has a predetermined length established outside of the body of the patient.

104. The method of claim 101, further comprising determining a length of the filament outside of the body of the patient prior to introducing the catheter into the lumenal system of the patient.

105. The method of claim 104, wherein the length of the filament is determined by using an imaging technique.

106. The method of claim 105, wherein the imaging technique is selected from the group consisting of echocardiography and fluoroscopy.

107. The method of claim 106, wherein the first region is a valve leaflet.

108. The method of claim 107, wherein the second region is a papillary muscle.

109. The method of claim 107, wherein the valve leaflet is located on the patient's tricuspid valve.

110. A system for implanting a filament in a lumenal system of a patient; comprising:
a) a catheter having an elongate body, the elongate body having a proximal end and a distal end;
b) an elongate filament having a predetermined length, the elongate filament having a first means for attachment to tissue at a first portion thereof and a second means for attachment to tissue at a second portion thereof

111. The system of claim 110, wherein the first means for attachment is adapted and configured to connect the first portion of the filament to a valve leaflet of a patient.

112. The system of claim 111, wherein the second means for attachment is adapted and configured to connect the second portion of the filament to cardiac tissue of a patient.

113. The system of claim 112, wherein the cardiac tissue is a papillary muscle head of the patient.

114. The system of claim 110, wherein at least one of the first means for attachment and second means for attachment include at least one barb for anchoring into tissue.
